SEQUENCE LISTING (2 sequences)

SEQ ID NO: 1 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 2 - DNA, 3366 bp, Homo sapiens
gcatgcgtaggcgcgcggccgcggcggcggctggggagggttcttccggaaggttcgggaggcttctggaaaaagcgccgcgcgctgggcgggcccgtcgctatataaggcaggcgcgggggtggcgcgtcagttgcttcagcgtcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaacatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggtttattttcacagattggggtaacgtgcactgtaagacgtatgtaacatgatgttaactttgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaaaataactcagacccagtcttgtggatggaaatgtagtgctcgagtcacattctgcttaaagttgtaacaaatacagatgagttaaaagatattgtgtgacagtgtcttatttagggggaaaggggagtatctggatgacagttagtgccaaaatgtaaaacatgaggcgctagcaggagatggttaaacactagctgctccaagggttgacatggtcttcccagcatgtactcagcaggtgtggggtggagcacacgtaggcacagaaaacaggaatgcagacaacatgcatcccctgcgtccatgagttacatgtgttctcttagtgtccacgttgttttgatgttattcatggaataccttctgtgttaaatacagtcacttaattccttggccttaaaa

(19) United States
(12) Patent Application Publication
(10) Pub. No.: US 2023/0321276 A1
(43) Pub. Date: Oct. 12, 2023

(54) NUCLEIC ACID THERAPY FOR DIFFERENTIAL MODULATION OF HOST MICROFLORA

(71) Applicant: ALMA BIO THERAPEUTICS, Lyon (FR)

(72) Inventors: Binah BAUM, Lyon (FR); Irun COHEN, Lyon (FR); Raanan MARGALIT, Lyon (FR)

(73) Assignee: ALMA BIO THERAPEUTICS, Lyon (FR)

(21) Appl. No.: 18/040,803

(22) PCT Filed: Sep. 29, 2021
(86) PCT No.: PCT/IB2021/058884

§ 371 Date: Feb. 6, 2023

Related U.S. Application Data
(60) Provisional application No. 63/085,163, filed on Sep. 30, 2020.

(51) Int. Cl. A61K 48/00; A61K 45/06; A61P 1/00; A61K 38/17

(52) U.S. Cl. CPC A61K 48/005; A61K 38/1709; A61K 45/06; A61P 1/00

(57) ABSTRACT

The invention is directed to nucleic acid therapy for modulating host microflora, useful in the management of dysbiosis. The invention in embodiments thereof provides compositions and methods for alleviating dysbiosis and conditions associated therewith. According to additional embodiments, compositions and methods of the invention may be used for treating or preventing gut barrier dysfunction in a subject in need thereof, and for reducing the risk of developing adverse events related to expansion of gastrointestinal bacteria in patients at risk for developing dysbiosis, for example in hospitalized patients and immune suppressed subjects.

### FIELD OF THE INVENTION

The invention is directed to nucleic acid therapy for modulating host microflora, useful in the management of dysbiosis.

### BACKGROUND OF THE INVENTION

The human gastrointestinal (GI) tract, the largest microbial reservoir in the body, harbors about 10¹⁴ microorganisms, predominantly bacteria. These microorganisms are collectively referred to as microbiota, while their collective genomes constitute what is known as the microbiome. The normal microbiota typically consist of 500-1000 different species, primarily inhabiting the colon, of which Firmicutes and Bacteroidetes species represent more than 90%. GI bacterial populations vary in their qualitative composition and abundance from the proximal to the distal portion, and from inner to outer lumen, and are influenced by the subject’s age, dietary habits, geographical origin, type of birth, antibiotic therapies, and exposure to environmental stimuli.

The gut microbiota is involved in a number of physiological functions including digestion, metabolism, extraction of nutrients, synthesis of vitamins, prevention of pathogen colonization, and immune modulation. Alterations or changes in composition and biodiversity of the gut microbiota have been observed in various metabolic states and in many gastrointestinal disorders and other pathophysiological conditions. For example, microbiota alterations have been associated or correlated with cases of obesity, celiac disease, irritable bowel syndrome (IBS), colon cancer, liver disorders, inflammatory bowel disease (IBD), diabetes, cystic fibrosis and allergies. Thus, specific microbiota profiles have been evaluated as markers for various pathophysiological states (Litvak et al., 2017, Current opinion in microbiology 39: 1-6; Mukhopadhya et al. 2012, Nature reviews Gastroenterology & hepatology 9.4: 219). At present, the interplay between the development of such pathologies and changes in microbiota composition and diversity is yet to be fully elucidated. Thus, studies aimed at investigating this interplay, attempting to determine whether a particular microbial alteration results from the development of a pathology of interest, and whether (and to what extent) it contributes to its etiology, are emerging as an intriguing field of study.

Heat shock proteins (HSPs) are evolutionarily conserved proteins that work as molecular chaperones for the intracellular transport and assistance during protein folding, helping in the degradation of unrecoverable denatured proteins. Situations of stress and/or pathologic conditions (such as inflammation) also influence the expression and activities of HSPs. For example, an aberrant expression of HSPs has been implicated in the pathogenesis of some autoimmune diseases.

It was reported that DNA vaccination with plasmids encoding HSPs such as mammalian HSP60, HSP70 or HSP90 showed beneficial effects in animal models of certain autoimmune diseases, in particular systemic lupus erythematosus (SLE), rheumatoid arthritis (RA) and type I diabetes (e.g. Liu et al. 2020, Arthritis Res Ther.;22(1):152; Quintana et al. 2004, Arthritis & Rheumatism, 50: 3712-3720). WO 03/096967, to some of the inventors of the present invention, relates to recombinant constructs encoding heat shock proteins or active fragments thereof, used as DNA vaccines for treating T cell mediated inflammatory autoimmune diseases. The recombinant constructs may encode e.g. HSP60, HSP70 or HSP90. The autoimmune disease may include RA, collagen II arthritis, multiple sclerosis, autoimmune neuritis, SLE, psoriasis, juvenile onset diabetes, Sjogren’s disease, thyroid disease, sarcoidosis, autoimmune uveitis, inflammatory bowel disease (Crohn’s and ulcerative colitis) or autoimmune hepatitis. WO 2018/178767, to some of the inventors of the present invention, provides methods and compositions for treatment of an HLA- B27-associated autoimmune inflammatory disorder by administration of nucleic acids encoding HSP90 or an active fragment thereof.

Certain HSPs, namely HSP25 (or HSP27, depending on the host species) and HSP70, are induced specifically in gut epithelial cells, and are thus defined as inducible HSPs (iHSPs). HSP25 and HSP70 are upregulated in gut epithelial cells in the presence of certain GI bacteria or their components, and confer protection against microbial toxins. However, whether iHSPs contribute to shape the gut microbiota, and whether additional HSPs may be involved, is presently unknown.

Various approaches of modulating the gut microbiota have been reported. Such approaches include for example probiotics (administration of live microorganisms), prebiotics (digestion-resistant dietary supplements that selectively enhance the growth and/or activity of a particular resident gut microbe), antimicrobial agents (e.g. antibiotics), as well as more indirect approaches such as surgery and weight loss strategies. In view of evidence that links the disruption in the composition and diversity of the gut microbiota to the development of certain pathologies, approaches aimed at modulating the gut microbiota have been suggested as potential therapies. Currently, in cases of severe or refractory dysbiosis or infection, the disease may only be manageable, if at all, by fecal microbiota transplantation (FMT) which is an unrefined bacteriotherapy that has many drawbacks and challenges relating inter alia to donor selection and sample handling, as well as risks of infection and toxins pass.

The ability to control uptake across the mucosa and to protect the gut from harmful substances present in the lumen is defined as intestinal barrier function. Compromised barrier integrity, or gut barrier dysfunction, is characterized by an increased intestinal permeability associated with GI dysbiosis, and is linked with pathological conditions. Gut barrier dysfunction can result, for example, from toxins, poor diet, parasites, infection, inflammation or medications, and may be found in patients with e.g. obesity or metabolic disorders. Critically ill patients and patients receiving chemotherapy and/or radiotherapy also show severely compromised intestinal barrier integrity. The clinical manifestations of gut barrier dysfunction may also be referred to as “leaky gut”.

There exists an unmet medical need for effective treatments specifically aimed at improving or restoring the gut microbiota ecosystem. In addition, there remains a need for novel therapeutic approaches for alleviating dysbiosis and conditions involving gut flora imbalance as part of their etiology.

### SUMMARY OF THE INVENTION

The invention is directed to nucleic acid therapy for modulating host microflora, useful in the management of dysbiosis. The invention in embodiments thereof provides compositions and methods for alleviating dysbiosis and conditions associated therewith. According to additional embodiments, compositions and methods of the invention may be used for treating or preventing gut barrier dysfunction in a subject in need thereof, and for reducing the risk of developing adverse events related to expansion of gastrointestinal bacteria in patients at risk for developing dysbiosis, for example in hospitalized patients and immune suppressed subjects.

The invention is based, in part, on the surprising discovery that administration of a nucleic acid construct encoding human heat shock protein 90 (HSP90) is capable of inducing specific modulation of intestinal microflora in vivo. Unexpectedly, intramuscular injection of the plasmid to C57BL/6 mice resulted in a specific reduction in the abundance of gut bacteria known to be associated with dysbiosis-related pathology. In particular, the relative abundance of facultative anaerobic Proteobacteria including Enterobacteriaceae Spp., and of facultative anaerobic Firmicutes taxa such as Enterococcaceae Spp., was specifically reduced. Further, administration of the plasmid surprisingly inhibited the development of dysbiosis in vivo. In particular, TNBS-induced expansion of Proteobacteria (e.g. Enterobacteriaceae, Escherichia) Spp, Bacteroidetes (Bacteroidaceae, Bacteroides) Spp, and facultative anaerobic Firmicutes (e.g. Enterococcaceae, Enterococcus) Spp., was inhibited in mice administered with the HSP90-expressing plasmid compared to those receiving a control plasmid.

Concomitantly, an increase (or inhibition in TNBS-induced reduction) in the abundance of bacterial strains known to exert beneficial effects in the context of gastrointestinal inflammation and/or gut barrier integrity, was unexpectedly observed. In particular, the relative abundance of obligate (obligatory) anaerobic Firmicutes-Clostridia (e.g. Lachnospiraceae spp., Clostridiales spp., Coprococcus spp., Ruminococcus spp.), Firmicutes-Bacilli-Lactobacillales (e.g. bactobacillaceae, L. reuteri), Bifidobacteriaceae (Bifidobacterium) spp. and Turicibacteraceae (Turicibacter) spp., was elevated.

Accordingly, embodiments of the invention are directed to methods for treating or preventing dysbiosis and/or for treating or preventing gut barrier dysfunction in a subject in need thereof. The methods of the invention in embodiments thereof comprise administering to the subject a nucleic acid construct comprising a nucleic acid sequence encoding a mammalian HSP90, or an active fragment thereof, wherein the nucleic acid sequence is operatively linked to one or more transcription control sequences.

In one aspect, there is provided a method of treating or preventing dysbiosis in a subject in need thereof, comprising administering to the subject a nucleic acid construct comprising a nucleic acid sequence encoding a mammalian HSP90, or an active fragment thereof, wherein the nucleic acid sequence is operatively linked to one or more transcription control sequences, to thereby treat or prevent dysbiosis in said subject.

In one embodiment, the dysbiosis is dysbiosis of the gastrointestinal (GI) tract. In another embodiment said dysbiosis is characterized by expansion of at least one of Bacteroidaceae, Enterobacteriaceae and Enterococcaceae. In yet another embodiment said dysbiosis is characterized by expansion of at least one of Bacteroidaceae, Enterobacteriaceae, Enterococcaceae and Porphyromonadaceae. In another embodiment said dysbiosis is characterized by expansion of facultative anaerobic Proteobacteria.

In another embodiment, treatment comprises inhibiting the growth of detrimental microbiota in the GI tract. In another embodiment, the detrimental microbiota comprise at least one pathogen species belonging to the Bacteroidaceae, Enterobacteriaceae and/or Enterococcaceae family. In another embodiment, the detrimental microbiota comprise at least one pathogen species belonging to the Bacteroidaceae, Enterobacteriaceae, Enterococcaceae and/or Porphyromonadaceae family. In another embodiment, said detrimental microbiota comprise at least one Enterococcus, Escherichia or Bacteroides species. In another embodiment said detrimental microbiota comprise at least one Enterococcus, Escherichia, Bacteroides or Parabacteroides species. In another embodiment said detrimental microbiota comprise at least one Enterococcus species, at least one Escherichia species and at least one Bacteroides species. In another embodiment, said detrimental microbiota comprise at least one Enterococcus species, at least one Escherichia species, at least one Bacteroides species and at least one Parabacteroides species.

In another embodiment treatment comprises promoting the growth of beneficial microbiota in the GI tract. In another embodiment the beneficial microbiota comprise at least one obligate anaerobic Firmicutes or bifidobacteriaceae species. In another embodiment the beneficial microbiota comprise at least one Firmicutes species and at least one bifidobacteriaceae species. In another embodiment said beneficial bacteria comprise at least one Lachnospiraceae, Lactobacillus, Clostridiales, Coprococcus, Ruminococcus and/or Turicibacter species. In another embodiment the method is used for modifying microbiota biodiversity in the GI tract. In another embodiment treatment comprises retaining or restoring the dominance of obligate anaerobic GI bacteria.

In another embodiment of the methods of the invention, the subject is afflicted with a disease or disorder associated with a bacterial, viral or parasitic infection or overgrowth, wherein each possibility represents a separate embodiment of the invention. In a particular embodiment the infection or overgrowth is bacterial (e.g. overgrowth of detrimental bacteria as disclosed herein). In another embodiment the disease or disorder is associated with infection by drug-resistant bacteria. In another embodiment the drug-resistant bacteria are selected from the group consisting of Enterococcus, Escherichia and Bacteroides species. In another embodiment said drug-resistant bacteria are selected from the group consisting of detrimental bacteria as disclosed herein, e.g. Proteobacteria, Gammaproteobacteria, Enterobacteriales Enterobacteriaceae and/or Escherichia species. In another embodiment said disease or disorder is a non-autoimmune inflammatory disorder of the GI tract.

In another embodiment of the methods of the invention, the subject is human. In another embodiment said subject exhibits dysbiosis. In another embodiment said subject is at risk for developing dysbiosis, e.g. due to exposure to antibiotics, immunosuppressants, chemotherapy or other treatments, conditions or procedures (e.g. surgery) associated with an enhanced risk for developing dysbiosis. In another embodiment said subject is under a treatment regimen with at least one of antimicrobial agents, parenteral nutrition, or immune suppressive agents. In another embodiment said subject is hospitalized, e.g. patients at intensive care units (e.g. due to acute cerebral infarction, severe burn damage or other critical care patients) with or prone to nosocomial infections.

In another embodiment, said dysbiosis is associated with gut barrier dysfunction. In another embodiment, said dysbiosis is associated with a GI disorder selected from the group consisting of: irritable bowel syndrome (IBS), celiac disease, small intestinal bacterial overgrowth (SIBO), leaky gut syndrome, and diverticular disease. In another embodiment said GI disorder is selected from the group consisting of: IBS, celiac disease, SIBO, leaky gut syndrome, diverticular disease, and AIDS enteropathy. In another embodiment said GI disorder is selected from the group consisting of: IBS, SIBO, leaky gut syndrome, and diverticular disease. Each possibility represents a separate embodiment of the invention. In other embodiments the dysbiosis is associated with e.g. a chronic lung disease, obesity, hypertension or metabolic diseases associated with expansion of proteobacteria.

In another embodiment of the methods of the invention, the construct is administered in combination with at least one antibiotic, probiotic or prebiotic agent. For example, without limitation, the antibiotic agent may be selected from nitroimidazoles, macrolides, and beta-lactams. In another embodiment, the probiotic agent comprises at least one of lactobacillus paracasei, lactobacillus casei, lactobacillus plantarum, lactobacillus acidophilus, lactobacillus reuteri and lactobacillus rhamnosus. In another embodiment the probiotic agent is a fecal microbiota transplant (FMT). In another embodiment the prebiotic agent is selected from the group consisting of lactulose, lignin, cellulose, hemicelluloses, β-glucans, pectin, gums, resistant starch, dextrin, psyllium, inulin, fructooligosaccharides, and polydextrose. In another embodiment the construct is administered in combination with a diet selected from the group consisting of: fiber-enriched, fructose-reduced, elemental, SIBO-specific, total liquid enteral, total parenteral, and peripheral parenteral diet.

In another embodiment the construct administered in connection with the methods of the invention encodes human HSP90. In another embodiment said construct encodes human HSP90 alpha. In another embodiment said construct is administered in the form of a pharmaceutical composition further comprising a pharmaceutically acceptable carrier, excipient and/or diluent. In another embodiment, the construct is administered in the form of a naked DNA (which may also be referred to as a naked DNA vaccine). In various embodiments, the construct may be administered by a route selected from the group consisting of parenteral, oral, topical and transdermal. In a particular embodiment, the administering is carried out parenterally. In another embodiment said administration is performed in a manner compatible with uptake of said construct by tissue-residing antigen presenting cells (APC), e.g. intramuscular or intradermal dendritic cells (DC). In another embodiment, said administration is performed intramuscularly. In a particular embodiment, said construct is administered by intramuscular injection. In another embodiment, said administration is performed subcutaneously.

In another embodiment said subject is further afflicted with a disease or condition resistant to an immunomodulatory treatment selected from immune suppressive treatment and anti-cytokine immunomodulatory treatment. In another embodiment said construct is administered in concurrent or sequential combination with a TNF-α antagonist or inhibitor. In another embodiment the TNF-α antagonist or inhibitor is selected from the group consisting of adalimumab, certolizumab, certolizumab pegol, golimumab, infliximab and etanercept. Each possibility represents a separate embodiment of the invention.

In another aspect, there is provided a method of treating or preventing gut barrier dysfunction in a subject in need thereof, comprising administering to the subject a nucleic acid construct comprising a nucleic acid sequence encoding a mammalian HSP90, or an active fragment thereof, wherein the nucleic acid sequence is operatively linked to one or more transcription control sequences.

In another embodiment, the dysfunction is associated with expansion of at least one of Bacteroidaceae, Enterobacteriaceae and Enterococcaceae. In yet another embodiment said dysfunction is characterized by expansion of at least one of Bacteroidaceae, Enterobacteriaceae, Enterococcaceae and Porphyromonadaceae. In another embodiment, said dysfunction is characterized by expansion of facultative anaerobic Proteobacteria. In another embodiment, said dysfunction is associated with expansion of a detrimental bacterial species as disclosed herein.

In another embodiment, the dysfunction is associated with a condition as disclosed herein, and/or the subject is afflicted with a disease or disorder as disclosed herein. In another embodiment, the subject is characterized and/or selected as disclosed herein. In another embodiment the construct is characterized and/or administered in a manner as disclosed herein. Each possibility represents a separate embodiment of the invention.

In another embodiment, said subject is afflicted with a GI disorder selected from the group consisting of: IBS, celiac disease, SIBO, leaky gut syndrome, and diverticular disease. In another embodiment, the construct is administered in combination with at least one antibiotic, probiotic or prebiotic agent. In another embodiment, the probiotic agent comprises at least one of lactobacillus paracasei, lactobacillus casei, lactobacillus plantarum, lactobacillus acidophilus, lactobacillus reuteri and lactobacillus rhamnosus. In another embodiment, the prebiotic agent is selected from the group consisting of lactulose, lignin, cellulose, hemicelluloses, β-glucans, pectin, gums, resistant starch, dextrin, psyllium, inulin, fructooligosaccharides, and polydextrose. In another embodiment, the construct is administered in combination with a diet selected from the group consisting of: fiber-enriched, fructose-reduced, elemental, total liquid enteral, SIBO-specific total parenteral, and peripheral parenteral diet.

In another embodiment, said construct encodes human HSP90 alpha. In another embodiment, said construct is administered in the form of a pharmaceutical composition further comprising a pharmaceutically acceptable carrier, excipient and/or diluent. In another embodiment, the construct is administered in the form of a naked DNA. In another embodiment, said construct is administered by intramuscular injection.

In another embodiment, said subject is further afflicted with a disease or condition resistant to an immunomodulatory treatment selected from immune suppressive treatment and anti-cytokine immunomodulatory treatment. In another embodiment, said construct is administered in concurrent or sequential combination with a TNF-α antagonist or inhibitor. In another embodiment, the TNF-α antagonist or inhibitor is selected from the group consisting of adalimumab, certolizumab, certolizumab pegol, golimumab, infliximab and etanercept. Each possibility represents a separate embodiment of the invention.

In another embodiment, the invention provides for determining whether a subject is amenable for treatment by an HSP90-encoding construct as disclosed herein, and for predicting or evaluating the therapeutic success. In some embodiment, a subject amenable for treatment by the methods of the invention exhibits dysbiosis characterized by expansion of detrimental bacteria as disclosed herein, e.g. facultative anaerobic Proteobacteria or at least one of Bacteroidaceae, Enterobacteriaceae and Enterococcaceae.

In another aspect, there is provided a method for treating an inflammatory GI disorder in a subject in need thereof, comprising: a) obtaining a sample comprising GI bacteria from the subject (e.g. a stool sample); b) determining the relative abundance of at least one of Bacteroidaceae, Enterobacteriaceae and Enterococcaceae (or in other embodiments, other detrimental bacteria as disclosed herein, e.g. Enterococcus, Escherichia or Bacteroides species), in the sample; c) comparing the abundance values measured to those corresponding to a healthy control, wherein significantly enhanced levels in the sample compared to the control sample indicate that said subject is amenable for the treatment, and d) administering to said subject, if determined amenable for said treatment, a nucleic acid construct comprising a nucleic acid sequence encoding a mammalian HSP90, or an active fragment thereof, wherein the nucleic acid sequence is operatively linked to one or more transcription control sequences.

In another embodiment, the sample is a stool sample. In another embodiment, wherein step b) comprises determining the relative abundance of Enterococcus, Escherichia and Bacteroides species in said sample. In another embodiment, the disorder is selected from the group consisting of: IBS, celiac disease, SIBO, leaky gut syndrome, and diverticular disease. In another embodiment, the disorder is resistant to an immunomodulatory treatment selected from immune suppressive treatment and anti-cytokine immunomodulatory treatment. In another embodiment, said construct is administered in concurrent or sequential combination with a TNFα antagonist or inhibitor. In another embodiment, the TNF-α antagonist or inhibitor is selected from the group consisting of adalimumab, certolizumab, certolizumab pegol, golimumab, infliximab and etanercept. Each possibility represents a separate embodiment of the invention.

Other objects, features and advantages of the present invention will become clear from the following description and drawings.

### BRIEF DESCRIPTION OF THE DRAWINGS

FIG. 1 is a schematic representation of the HSP90 plasmid (pcDNA3.1-HSP90).

FIG. 2 presents the experimental treatment scheme. D0 (day 0) indicates the day of TNBS intrarectal administration. The arrows indicate intramuscular administration of HSP90 plasmid, control plasmid or vehicle.

FIG. 3 is a boxplot representing the proportion (relative abundance) of Proteobacteria in each group. CTL - no treatment; CTL_HSP90 - HSP90 plasmid only; TNBS_PBS - TNBS treated mice, no plasmid; TNBS_emptyPlasm - TNBS treated mice, empty vector control plasmid; TNBS_HSP90 - TNBS treated mice further receiving the HSP90 plasmid.

FIG. 4 is a boxplot representing the proportion of Enterobacteriaceae in each group.

FIG. 5 is a boxplot representing the proportion of Escherichia in each group.

FIG. 6 is a boxplot representing the proportion of Bacteroidaceae in each group.

FIG. 7 is a boxplot representing the proportion of Bacteroides in each group.

FIG. 8 is a boxplot representing the proportion of Enterococcaceae in each group.

FIG. 9 is a boxplot representing the proportion of Enterococcus in each group.

FIG. 10 is a boxplot representing the proportion of Bifidobacterium in each group.

FIG. 11 is a boxplot representing the proportion of bifidobacteriaceae in each group.

FIG. 12 is a boxplot representing the proportion of Turicibacter in each group.

FIG. 13 is a boxplot representing the proportion of Turicibacteraceae in each group.

FIGS. 14A-14E are boxplots representing the proportion of different Clostridiales operational taxonomic units (OTU) in each group of the second experiment.

FIGS. 15A-15D are boxplots representing the proportion of specific Firmicutes OTU in each group of the second experiment: FIG. 15A - Lachnospiraceae; FIG. 15B - Lactobacillus reuteri; FIG. 15C - Lachnospiraceae-Coprococcus; and FIG. 15D - Lachnospiraceae-Ruminococcus.

### DETAILED DESCRIPTION OF THE INVENTION

The present invention is directed to the modulation of gut microbiota. Specifically, the invention relates to compositions and methods for restoring gastrointestinal homeostasis, for modifying gastrointestinal microbiota profiles at various levels of the gastrointestinal (GI) tract, and for alleviating gastrointestinal disorders and other conditions associated with imbalance of gut flora. According to additional embodiments, compositions and methods of the invention may be used for treating or preventing gut barrier dysfunction in a subject in need thereof, and for reducing the risk of developing adverse events related to expansion of gastrointestinal bacteria in patients at risk for developing dysbiosis, for example in hospitalized patients and immune suppressed subjects.

The invention is based, in part, on the surprising discovery that administration of a nucleic acid construct encoding human heat shock protein 90 (HSP90) is capable of inducing differential modulation of intestinal microflora in vivo, and exerts beneficial therapeutic effects in the context of dysbiosis management. As disclosed herein, the modulations further included up-regulation of bacteria exerting protective or supportive effects on gut barrier function, and down-regulation of bacteria exerting detrimental effects on gut barrier function or integrity.

Accordingly, the invention provides in some embodiments for the treatment of new patient populations, not hitherto considered amenable for treatment with immunomodulating therapy, or with DNA vaccines encoding heat shock proteins.

In one aspect, there is provided a method of treating or preventing dysbiosis in a subject in need thereof, comprising administering to the subject a nucleic acid construct comprising a nucleic acid sequence encoding a mammalian HSP90, or an active fragment thereof, wherein the nucleic acid sequence is operatively linked to one or more transcription control sequences, to thereby treat or prevent dysbiosis in said subject.

In another aspect, there is provided a method of treating or preventing gut barrier dysfunction in a subject in need thereof, comprising administering to the subject a nucleic acid construct comprising a nucleic acid sequence encoding a mammalian HSP90, or an active fragment thereof, wherein the nucleic acid sequence is operatively linked to one or more transcription control sequences.

In another aspect, there is provided a method for determining whether a subject in need thereof (e.g. a subject as described herein) is amenable for treatment with a nucleic acid construct comprising a nucleic acid sequence encoding a mammalian HSP90, or an active fragment thereof, wherein the nucleic acid sequence is operatively linked to one or more transcription control sequences, the method comprising:
a) obtaining a sample comprising GI bacteria from the subject (e.g. a stool sample);
b) determining the relative abundance of at least one of Bacteroidaceae, Enterobacteriaceae and Enterococcaceae (or in other embodiments, other detrimental bacteria as disclosed herein, e.g. Enterococcus, Escherichia or Bacteroides species), in the sample; and
c) comparing the abundance values measured to those corresponding to a healthy control, wherein significantly enhanced levels in the sample compared to the control sample indicate that said subject is amenable for treatment with said construct.

In yet another aspect, the invention relates to a method for treating an inflammatory GI disorder in a subject in need thereof, comprising:
a) obtaining a sample comprising GI bacteria from the subject (e.g. a stool sample);
b) determining the relative abundance of at least one of Bacteroidaceae, Enterobacteriaceae and Enterococcaceae (or in other embodiments, other detrimental bacteria as disclosed herein, e.g. Enterococcus, Escherichia or Bacteroides species) in the sample;
c) comparing the abundance values measured to those corresponding to a healthy control, wherein significantly enhanced values compared to the control sample indicate that said subject is amenable for treatment, and
d) administering to said subject, if determined amenable for treatment, a nucleic acid construct comprising a nucleic acid sequence encoding a mammalian HSP90, or an active fragment thereof, wherein the nucleic acid sequence is operatively linked to one or more transcription control sequences.

### Microbiota

The gut microbiota constituents, functions, homeostasis, and interactions with the host play a crucial role in health maintenance, preventing pathogen colonization, and contributing to the maturation and education of the immune system. Imbalance of the microbiota composition and/or diversity may lead to (or accompany) a variety of diseases or disorders.

In some embodiments, the methods of the invention comprise a therapeutic effect on microbiome in a subject. In some embodiments, the methods of the invention modulate gut microbiota. In certain exemplary embodiments, the methods of the invention result in the increasing of number and/or types of bacteria that are beneficial to the gut. In additional embodiments, the methods of the invention result in the reduction of number and/or types of bacteria that are detrimental to the gut.

In some embodiments, the compositions and methods of the present disclosure treat or prevent dysbiosis in a subject. In other embodiments, the subject to be treated by the compositions and methods of the invention exhibits dysbiosis, or is at risk for developing dysbiosis.

The term “dysbiosis” refers to a refers to a state of the microbiota of the gut or other body area in a subject, in which the normal diversity and/or function of the microbial populations is disrupted. The term encompasses imbalances in quality, absolute quantity, or relative quantity of members of the microbiota of a subject, and is characterized by differences in one or more of these parameters compared to a healthy control subject. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health. According to non-limitative examples, essential functions may include enhancement of the gut mucosal barrier integrity, direct or indirect reduction and elimination of invading pathogens, enhancement of the absorption of specific substances, and suppression of GI inflammation.

In some embodiments, dysbiosis referred to in embodiments of the invention is GI dysbiosis. In a particular embodiment, the dysbiosis is at a particular location of the GI tract, e.g. the colon. In another specific embodiment, the dysbiosis is oral dysbiosis.

In some embodiment the dysbiosis is characterized by expansion of at least one of Bacteroidaceae, Enterobacteriaceae and Enterococcaceae. In yet another embodiment said dysbiosis is characterized by expansion of at least one of Bacteroidaceae, Enterobacteriaceae, Enterococcaceae and Porphyromonadaceae. In another embodiment said dysbiosis is characterized by expansion of facultative anaerobic Proteobacteria. In some embodiments, the methods described herein result in the reduction of the number of at least one detrimental bacterial strain.

The term “expansion” when relates to bacteria refers to a significant increase in the number or abundance of said bacteria at a particular location or habitat (e.g. the GI tract or a section thereof). Bacterial expansion (also referred to herein as bacterial growth) may be attributed to enhanced proliferation, reduced death rate, enhanced colonization, or combinations thereof. In some embodiments, dysbiosis characterized by expansion of a microorganism denotes enhancement in said microorganism levels as compared to their levels in the absence of dysbiosis, e.g. in the same patient prior to the development of dysbiosis or in a healthy control subject.

The term “facultative anaerobic” refers to a microorganism that can grow in both aerobic and anaerobic environments.

Bacteroidaceae is a family of environmental bacteria that are common in the human gastrointestinal microbiota. It is a family of gram-negative bacteria found primarily in the intestinal tracts and mucous membranes of warm-blooded animals and are predominate in infections that originate from the gut flora.

Enterobacteriaceae is a family of gram-negative bacteria that include many of the more familiar pathogens, such as Klebsiella, Salmonella, Shigella and Escherichia coli. Members of the Enterobacteriaceae are bacilli (rod-shaped), facultative anaerobes, fermenting sugars to produce lactic acid and various other end products.

Enterococcaceae is a family of Gram-positive bacteria placed in the order Lactobacillales. The family includes various cocci strains (notably belonging to the genus Enterococcus) that are among the most common nosocomial pathogens that could cause infections and diseases including, but not limited to bacteremia, urinary, intra-abdominal and pelvic infections.

The genus Enterococcus includes gram-positive bacteria that often occur in pairs (diplococci) or short chains. Two species that are common in the intestines of humans are E. faecalis (90-95%) and E. faecium (5-10%). Additional clusters of infections occur with other species, including, but not limited to, E. casseliflavus, E. gallinarum, and E. raffinosus.

Escherichia is a genus of Gram-negative, non-spore-forming, facultatively anaerobic, rod-shaped bacteria from the family Enterobacteriaceae.

Bacteroidetes and Proteobacteria are enriched in the luminal content of the gut. Bacteroides is a genus of Gram-negative, obligate anaerobic bacteria. Bacteroides species are non-endospore-forming bacilli, and may be either motile or nonmotile, depending on the species. Certain Bacteroides species are normally mutualistic, making up the most substantial portion of the mammalian gastrointestinal microbiota, where they play a role in processing of complex molecules (e.g., complex carbohydrates) to simpler ones in the host intestine and producing favorable metabolites. Other Bacteroides are significant clinical pathogens and are found in most anaerobic infections. These detrimental bacteria maintain a complex and generally beneficial relationship with the host when retained in the gut, but when they escape this environment they can cause significant pathology, including bacteremia and abscess formation in multiple body sites. For example, B. fragilis, which accounts for only 0.5% of the human colonic flora, is the most commonly isolated anaerobic pathogen due, in part, to its potent virulence factors.

The Porphyromonadaceae is a family within the order Bacteroidales, comprises the genera Porphyromonas, Barnesiella, Butyricimonas, Dysgonomonas, Macellibacteroides, Odoribacter, Paludibacter, Parabacteroides, Petrimonas, Proteiniphilum, and Tannerella.

Proteobacteria is a major phylum of Gram-negative bacteria that include various pathogenic genera, such as Klebsiella, Escherichia, Salmonella, and Campylobacter. Proteobacteria are usually associated with imbalance of microbiota of the gut and may serve as a possible microbial signature of disease.

The term “pathogen” or “pathogenic” in reference to a bacterium or any other organism or entity includes any such organism or entity that is capable of causing or promoting a disease, disorder or condition of a host organism containing the organism or entity. In contradistinction from acquired infectious pathogens, a “pathobiont” is a potentially disease-causing member of the microbiota that is present in the microbiota of a non-diseased or a diseased subject, and which has the potential to contribute to the development or progression of a disease or disorder. Under normal circumstances, pathobionts may live as non-harming commensals or symbionts, but may trigger immune-mediated pathology and/or disease in response to certain genetic or environmental factors. The term pathogen encompasses both acquired infectious organisms, as well as pathogenic pathobionts.

The term “detrimental bacterium” as used herein refers to bacterial pathogens and pathobionts. Non limitative examples of detrimental bacteria include e.g. Proteobacteria (e.g. Enterobacteriaceae, Escherichia) taxa, Bacteroidetes (Bacteroidaceae, Bacteroides) taxa, and facultative anaerobic Firmicutes (e.g. Enterococcaceae, Enterococcus) taxa. Exemplary detrimental taxa are further provided in Table 1 below.

TABLE 1

Exemplary detrimental bacterial taxa

| | | | | |
|---|---|---|---|---|
| Phylum/ Class | Order | Family | Genus | Species |
| Bacteroidetes/ Bacteroidia | Bacteroidales | Bacteroidaceae | Bacteroides | B. fragilis, B. vulgatus B. uniformis |
| Proteobacteria/ Gammaproteobacteria | Enterobacteriales | Enterobacteriaceae | Escherichia | E. albetii, E. coli, E. fergusonii, E. hermanii. E. vulneris |
| Firmicutes/ Bacilli | Lactobacillales | Enterococcaceae | Enterococcus | E. faecalis, E. faecium |

According to some embodiments, the treatments as described herein comprise promoting the growth of beneficial microbiota in the GI tract. In another embodiment, the beneficial microbiota comprise at least one obligate anaerobic Firmicutes or Bifidobacteriaceae species. In another embodiment, the beneficial microbiota comprise at least one Firmicutes species and at least one Bifidobacteriaceae species. In another embodiment said beneficial bacteria comprise at least one Lachnospiraceae, Clostridiales, Coprococcus, Ruminococcus, Lactobacillus, and/or Turicibacter species. In another embodiment the method is used for modifying microbiota biodiversity in the GI tract. In another embodiment treatment comprises retaining or restoring the dominance of obligate anaerobic GI bacteria.

The term “beneficial microbiota” refers to microorganisms that live in the digestive tract of a subject, for example a human or other animal, and have a positive effect on the health of the subject. The beneficial bacteria typically comprise one of more of Bifidobacteria and Lactobacilli. Studies have demonstrated effects of beneficial bacteria on e.g. intestinal inflammation, diarrhea, urogenital infections, allergies, blood pressure control, bacterial vaginosis, eczema, immune functions and infections. According to advantageous embodiments, beneficial microbiota referred to in the context of the methods of the invention exert beneficial effects on gastrointestinal inflammation and/or gut barrier integrity, and include, but not limited to, obligate anaerobic Clostridial Cluster XIVa (Firmicutes; Clostridia; Clostridiales; Lachnospiraceae - genera: Blautia, Coprococcus, Roseburia), and Clostridial cluster IV (Ruminococcus), Firmicutes-Bacilli-Lactobacillales (e.g. bactobacillaceae, L. reuteri), Bifidobacteriaceae (Bifidobacterium) taxa and Turicibacteraceae (Turicibacter) taxa. Additional exemplary beneficial Lactobacillus Spp. include, for example, L. acidophilus, L. gasseri, L. casei, L. plantarum, L. reuteri, and L. paracasei. Additional exemplary beneficial strains classified as Clostridial cluster IV include e.g. C. leptum, C. sporosphaeroides, C. cellulosi, and F. prausnitzii. Additional exemplary beneficial strains classified as Clostridial cluster XIVa include e.g. C. aerotolerans, and C. nexile.

The term “promoting the growth of” as used herein refers to activation, facilitation, increasing or enhancing the growth of what has been defined. Promoting bacterial growth leads to an enhancement in the absolute or relative abundance of the bacteria in question, as described herein.

In various particular embodiments, promoting or inhibiting bacterial growth may refer to enhancement (or reduction, respectively) of at least twofold, and typically at least eightfold, tenfold, 50-fold, 100-fold or more. Conveniently, the enhancement or reduction may be represented as the base 2 logarithm thereof (log2(fold change) or log2FC), and be characterized by a log2FC of e.g. -11 to -7, -10 to -8 or -9 to -5 (in case of reduction) or 2 to 12, 6 to 11 or 6.5 to 10 (in case of enhancement). For instance, as exemplified herein, administration of a nucleic acid construct encoding human HSP90 resulted in a reduction in Enterococcaceae counts and Enterobacteriaceae counts of -8.61 and -5.82 log2FC, respectively.

The term “obligate anaerobic bacteria” (also referred to as “obligatory anaerobic bacteria”) refers to microorganisms that are killed by normal atmospheric concentrations of oxygen (20.95% O₂). Obligate anaerobes convert nutrients into energy through anaerobic respiration or fermentation. During healthy homeostasis, about 97% of gut bacteria are obligate anaerobes, mostly belonging to the phyla Firmicutes (64%), Bacteroidetes (23%), Proteobacteria (8%), and Actinobacteria (3%). The term “dominance of obligate anaerobic GI bacteria” refers to a state of microbiota (or profile of GI microflora) that is substantially similar to the healthy homeostasis state or profile, as specified herein. In some embodiments, the dominance is characterized by at least 90% and typically at least 95%, 96% or 97%, more typically 95-97%, 96-97% or 96-98% obligate anaerobes in the GI tract.

The term “biodiversity” (or “diversity”) refers to the extent to which different taxonomic groups of microorganisms are present in a population of microorganism. In order to quantify and compare microbial taxonomic diversity, i.e., “within-sample diversity”, diversity calculation using the Shannon index or other acceptable methods may conveniently be applied.

0Firmicutes is a phylum of bacteria, most of which have gram-positive cell wall structure, that colonize the mucin layer. The Firmicutes make up the largest portion of the human gut microbiome and their relative abundance increases with age. Firmicutes phylum includes, for example, genera such as Lactobacillus, Bacillus, Clostridium, Enterococcus, and Ruminicoccus.

Bifidobacteriaceae is a family of bacteria, members of which are typical gut inhabitants, and represent non-motile, non-gas producing, saccharolytic Gram-positive bacteria.

Lachnospiraceae are a family of anaerobic, spore-forming bacteria in the order Clostridiales that ferment diverse plant polysaccharides to short-chain fatty acids and alcohols. These bacteria are among the most abundant taxa in the human gut microbiota.

Clostridium is a genus of Gram-positive bacteria, belonging to the family of Firmicutes. Clostridium is well known as a gut colonizer, which is found significantly in infants and adults.

Coprococcus is a genus of anaerobic cocci which are part of the normal flora of the mouth, upper respiratory tract, intestinal tract, vagina and skin.

Ruminococcus is a genus of bacteria in the class Clostridia. They are anaerobic, Gram-positive gut microbes.

Lactobacillus is a genus of Gram-positive, aerotolerant anaerobes or microaerophilic, rod-shaped, non-spore-forming bacteria. They constitute a major component of the microbiota in the body and are responsible for breaking down dietary fibers and producing beneficial substances, like short-chain fatty acids.

Turicibacter is a genus in the Firmicutes phylum of bacteria associated with intestinal butyric acid. An exemplary beneficial species is Turicibacter sanguinis.

### Heat Shock Protein 90 (HSP90) and Constructs Encoding Same

In general, the methods of the present disclosure involve administration of a nucleic acid encoding a heat shock protein 90 (HSP90) to a subject so that the HSP90-encoding nucleic acid provides for production of HSP90 protein in the host. Such constructs can be a DNA construct in which the HSP90-encoding DNA is operably linked to a promoter that facilitates expression in the subject.

The HSP90 encoded by the nucleic acid of the construct can be any suitable HSP90, or an active fragment thereof, in particular, a mammalian HSP90 or active fragment thereof, e.g., a human HSP90, rat HSP90, or mouse HSP90, or an active fragment thereof. The nucleic acid sequence encoding the HSP90may include DNA, RNA, or derivatives of either DNA or RNA with the proviso such are amenable to production of the encoded HSP90 protein gene product in a host cell, e.g., a human cell. The nucleic acid sequence encoding the HSP90 can be obtained from a natural source, either as encoding a full-length HSP90 or a portion thereof. A nucleic acid molecule can also be produced using recombinant DNA technology (e.g., polymerase chain reaction (PCR) amplification, cloning) or chemical synthesis. Nucleic acids encoding HSP90 include naturally-occurring HSP90-encoding nucleic acids and homologues thereof, including, but not limited to, natural allelic variants and modified nucleic acid sequences in which nucleotides have been inserted, deleted, substituted, and/or inverted in such a manner that such modifications do not substantially interfere with the nucleic acid molecule’s ability to provide for production a functional HSP90 or an active fragment thereof in a cell of the subject to whom it is administered.

Conserved/functional domains shared by all HSP90 proteins which may fulfill HSP90-specific functions have been identified (see for example, Chen et al., BMC Genomics, 7:156, 2006). These include for example an ATP-binding domain, an ATPase domain, a glutamic acid-rich motif; a four-helical cytokine region a lysine-rich domain, a protein kinase C phosphorylation site, a casein kinase II phosphorylation site, a N-glycosylation site, a tyrosine kinase phosphorylation site, a tyrosine sulfation site, a cAMP- and cGMP-dependent protein kinase phosphorylation site, an N-myristoylation site, a bipartite nuclear targeting site, a leucine zipper domain, and an amidation site.

As used herein, an active fragment of HSP90 denotes a truncated HSP90 polypeptide which retains at least the evolutionary conserved regions of HSP90, so as to retain its ability to modulate host microflora. In some embodiments, an HSP90 homolog or fragment is at least about 67% and typically at least about 70%, 80%, 85%, 90%, 95%, 98% or 99% homologous to a naturally occurring mammalian HSP90, e.g. human HSP90 as disclosed herein. For example, isoform 1 of a human HSP90 alpha polypeptide disclosed in connection with embodiments of the invention is identical to isoform 2 of a human HSP90 alpha polypeptide disclosed in connection with embodiments of the invention, with the exception that it contains additional 122 amino acids at its N-terminus. Thus, isoforms 1 and 2 of human HSP90 alpha are characterized by 85.7% overall homology, while being identical throughout the shared section that is conserved between the isoforms. Typically, the encoded polypeptide is characterized as retaining immunological cross-reactivity with mammalian and bacterial HSP (e.g. containing cross-reactive T-reg epitopes of mammalian and microbial HSP90).

Where the subject is human, a nucleic acid encoding a human HSP90 (either full- length or an active fragment thereof) is of particular interest. The human HSP90 can be either HSP90 alpha or HSP90 beta, wherein each possibility represents a separate embodiment of the invention. The amino acid sequences of examples of human HSP0 proteins suitable for use in the present methods include, for example, NCBI reference sequence NP 001017963.2 (heat shock protein HSP 90-alpha isoform 1) and NCBI reference sequence NP 005339.3 (heat shock protein HSP 90-alpha isoform 2). The amino acid sequence of an example of a full-length human HSP90 protein is provided below:

MPEETQTQDQPMEEEEVETFAFQAEIAQLMSLIINTFYSNKEIFLRELIS
NSSDALDKIRYESLTDPSKLDSGKELHINLIPNKQDRTLTIVDTGIGMTK
ADLINNLGTIAKSGTKAFMEALQAGADISMIGQFGVGFYSAYLVAEKVTV
ITKHNDDEQYAWESSAGGSFTVRTDTGEPMGRGTKVILHLKEDQTEYLEE
RRIKEIVKKHSQFIGYPITLFVEKERDKEVSDDEAEEKEDKEEEKEKEEK
ESEDKPEIEDVGSDEEEEKKDGDKKKKKKIKEKYIDQEELNKTKPIWTRN
PDDITNEEYGEFYKSLTNDWEDHLAVKHFSVEGQLEFRALLFVPRRAPFD
LFENRKKKNNIKLYVRRVFIMDNCEELIPEYLNFIRGVVDSEDLPLNISR
EMLQQSKILKVIRKNLVKKCLELFTELAEDKENYKKFYEQFSKNIKLGIH
EDSQNRKKLSELLRYYTSASGDEMVSLKDYCTRMKENQKHIYYITGETKD
QVANSAFVERLRKHGLEVIYMIEPIDEYCVQQLKEFEGKTLVSVTKEGLE
LPEDEEEKKKQEEKKTKFENLCKIMKDILEKKVEKVVVSNRLVTSPCCIV
TSTYGWTANMERIMKAQALRDNSTMGYMAAKKHLEINPDHSIIETLRQKA
EADKNDKSVKDLVILLYETALLSSGFSLEDPQTHANRIYRMIKLGLGIDE
DDPTADDTSAAVTEEMPPLEGDDDTSRMEEVD (human HSP90-alph
a isoform 2, SEQ ID NO: 1).

Any suitable constructs facilitating delivery of the HSP-encoding nucleic acid into a host cell, particularly a human host cell, and facilitating expression in the host cell, can be used. Suitable constructs include e.g. plasmids.

In some embodiments, the HSP90-enocoding construct comprises at least one CpG motif, which may be defined by the generic formula 5′-XiCGX₂-3′, wherein Xi and X₂ represent any nucleotide, and the central CG dinucleotide is unmethylated. Examples of CpG motif-containing sequences include nucleic acid having a sequence of the formulae 5′-RRCGYY-3′, 5-RTCGYY-3, 5-RRCGYYCG-3, 5-RTCGYYCG-3, wherein the “CG” are unmethylated CpG dinucleotides, R represents a purine (A or G) and Y represents a pyrimidine (C or T). Yet in other embodiments, the construct does not contain a CpG motif. In another embodiment said construct further encodes an antibiotic-resistance gene (e.g. an ampicillin-resistant cassette). In another embodiment said construct does not further encode an antibiotic-resistance gene. Each possibility represents a separate embodiment of the invention.

The HSP90-encoding constructs generally include a nucleic acid sequence encoding an HSP90 (or active fragment thereof), e.g., a human HSP90, operatively linked to one or more transcription control elements in an expression construct. Transcription control elements include nucleic acids having sequences that facilitate control of initiation, elongation, and termination of transcription. Transcription control elements include promoters, enhancers, operators and repressors. Of particular interest are transcription control elements that facilitate expression in a mammalian cell, particularly a human cell. Promoters of interest in the present constructs include constitutive promoters, inducible promoters, and tissue-specific promoters, with constitutive promoters being of particular interest. Examples of promoters that can find use in the constructs of the present of disclosure to provide for expression of HSP90 in a subject include, but at not necessarily limed to, viral promoters, such as CMV promoters, RSV promoters, retroviral promoters, and SV-40 promoters.

The phrase “operatively linked” refers to linking a nucleic acid sequence to a transcription control sequence in such a manner that the encoded protein molecule is able to be expressed when the nucleic acid sequence is transfected (i.e., transformed, transduced or transfected) into a host cell. Particularly important transcription control sequences are those which control transcription initiation, such as promoter, enhancer, operator and repressor sequences. In a particular embodiment, the nucleic acid sequence is operatively linked to a promoter sequence as disclosed herein.

As used herein, the term “nucleic acid construct” refers to a DNA or RNA molecule that comprises a polynucleotide sequence which encodes a protein of interest (e.g. HSP90), and which includes initiation and termination signals operably linked to regulatory elements including a promoter and polyadenylation signal capable of directing expression in the cells of a subject. Thus, a gene construct contains the necessary regulatory elements operably linked to the polynucleotide sequence that encodes the protein, such that when present in a cell of the individual, the polynucleotide sequence will be expressed. Examples of eukaryotic expression constructs for use in the methods of the present disclosure include: pcDNA3, pcDNA3.1 (+/-), pWRG7077, pZeoSV2(+/-), pSecTag2, pDisplay, pEF/myc/cyto, pCMV/myc/cyto, pCR3.1, pCI, pBK-RSV, pBK-CMV, pTRES or their derivatives.

Additional examples of promoters useful to practice embodiments of the invention include, but are not limited to, promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV), promoters from human genes such as human actin, human myosin, human hemoglobin, human muscle creatine and human metalothionein and tissue-specific promoters such as involucrin, keratin 5, and keratin 14. Suitable protocols for use of promoters in construction of gene constructs are well known in the art (see, for example, Current Protocols in Molecular Biology, Chapter 1 (Wiley Interscience, 1989)).

Examples of polyadenylation signals useful to practice the present invention include, but are not limited to, SV40 polyadenylation signals and LTR polyadenylation signals. Examples of enhancers include, but are not limited to, enhancers of human actin, human myosin, human hemoglobin, human muscle creatine and viral enhancers such as those from CMV, RSV and EBV.

Advantageously, constructs to be used in embodiments of the invention do not integrate into the subject’s genome. Nucleic acid constructs can be provided with mammalian origin of replication in order to maintain the construct extrachromosomally and produce multiple copies of the construct in the cell. For example, plasmids pCEP4 and pREP4 from Invitrogen (San Diego, Calif.) contain the Epstein Barr virus origin of replication and nuclear antigen EBNA-1 coding region, which produces high copy episomal replication without integration. Other plasmids known in the art may be used so long as the gene constructs express the HSP90 protein encoded by the polynucleotide sequence.

In a particular embodiment, said construct is an expression vector amenable for use as a DNA vaccine in clinical settings. For example, without limitation, an HSP90-encoding construct in accordance with embodiments of the invention may be produced based on the pWRG7077 backbone (Aldevron, Fargo, ND). In some embodiments, the construct lacks an antibiotic resistance gene, for example the kanamycin-resistance cassette of the pWRG7077 vector may be removed and/or replaced by an open reading frame encoding for another selection system (e.g. a selection marker such as RNA-OUT that represses the SacB gene in the engineered host cell, wherein growth of the cells and maintenance of plasmid copy number and selection pressure may be maintained by use of sucrose in the medium). In another exemplary embodiment, said construct backbone is further enriched by additional CpG sequences, as disclosed herein. It is to be understood, that the term “DNA vaccine” as used herein refers to a therapeutic DNA composition for the treatment and/or prevention of dysbiosis or a condition as disclosed herein in a human subject, and does not necessitate an immune response to be generated against a specific microorganism or pathogen.

Advantageously, said construct or vector is used in the form of a naked DNA. In a particular embodiment, said construct is in the form of a supercoiled DNA or supercoiled plasmid, typically a naked DNA supercoiled plasmid. The term “supercoil” is defined as the physical state of a polynucleotide in which one strand of the polynucleotide is underwound or overwound in relation to other strands of the polynucleotide. A supercoiled plasmid in accordance with the invention is in the form of a double-stranded DNA in a circular supercoiled form. Without wishing to be bound by a specific theory or mechanism of action, supercoiled DNA may afford higher shear-resistance than either the relaxed, open circular or nicked forms of the plasmid, due to its reduced size.

In another embodiment, other constructs, e.g. those based on viral vectors or RNA replicons, may be used, as long as these constructs retain the ability to express the encoded HSP90 molecule in a mammalian host cell, including in particular an antigen presenting cell (e.g. human dendritic cells or macrophages). According to a particular embodiment, targeted delivery and/or preferential expression in human dendritic cells (DC), or specific populations thereof (e.g. muscle DC, skin DC and/or intestinal DC) is contemplated. Various exemplary constructs and compositions for functional RNA delivery targeted to dendritic cells are described e.g. by McCullough et al., Ther Deliv. 2012 Sep;3(9):1077-99, which is incorporated herein by reference.

Viral vectors suitable for delivery in vivo and expression of an exogenous protein are well known and include adenoviral vectors, adeno-associated viral vectors, retroviral vectors, herpes simplex viral vectors, and the like. Viral vectors are preferably made replication defective in normal cells. See e.g. U.S. Pat. Nos. 6,669,942; 6,566,128; 6,794,188; 6,110,744; and 6,133,029.

### Pharmaceutical Compositions

The HSP90-encoding constructs can be provided in any formulation suitable for administration to a subject, e.g., suitable for administration of a human subject.

Compositions suitable for administration to a subject can include the HSP90- encoding construct and a pharmaceutically acceptable carrier. As used herein, “pharmaceutically acceptable carrier” includes any material which, when combined with an HSP-encoding construct of the present disclosure, retains the biological activity and preferably improves stability of the construct without causing significant adverse reactions. Examples include, but are not limited to, any of the standard carriers such as an isotonic solution of a pharmaceutically acceptable alkali metal salt (e.g., sodium chloride), a phosphate buffered saline (PBS) solution, water, Ringer’s solution, dextrose solution, emulsions such as oil/water emulsion, and various types of wetting agents. Aqueous carriers can contain suitable auxiliary substances required to approximate the physiological conditions of the recipient, for example, by enhancing chemical stability and isotonicity. Examples of additives of interest in formulation of the present disclosure include, but are not limited to, sodium citrate, malic acid, ethanol, and PLURONIC F-68® (poloxamer 188). Additional examples of additives include, for example, pharmaceutically acceptable salts (e.g., sodium acetate, sodium lactate, potassium chloride, calcium chloride), and preservatives. In general formulations are sterile.

Compositions of interest also include a sterile formulation comprising HSP90- encoding nucleic acid (e.g., HSP90-encoding DNA, e.g., in an expression construct operably linked to a promoter, such as a strong constitutive promoter (e.g., CMV)) formulated with 0.9% pharmaceutically acceptable alkali metal salt (e.g., sodium salt, e.g., NaCl).

HSP90-encoding nucleic acid can optionally be formulated with a lipid carrier, e.g., so as to be provided as a payload in a stabilized lipid particle, as in a complex with a cationic lipid, e.g., in a liposome or micelle. In such embodiments, the lipid carrier can be modified (e.g., by manipulating the chemical formula of the lipid portion of the delivery vehicle and/or providing a compound capable of targeting the lipid to a target site, for example, a target cell type so as to provide for interaction of lipid carrier (or, for example, other liposome component) with a molecule on the surface of the cell. Suitable targeting compounds include ligands capable of selectively binding another molecule at a particular site. Examples of such ligands include antibodies, antigens, receptors and receptor ligands.

HSP90-enoding nucleic acids can optionally be formulated with a suitable polymer, such as polyethylene glycol, polylysine, poloxamer, chitosan, polyL glutamate, poly(lactide-co-glycolide) (PLG).

In some embodiments, the HSP90-encoding nucleic acid is not encapsulated (e.g., within a liposome). In some the HSP90-encoding nucleic acid is not contained in a viral particle. In some embodiments, the nucleic acid is “naked”, i.e., is not encapsulated (e.g., within a liposome) and is not contained in a viral particle.

The HSP90-encoding nucleic acid construct may be provided in a sterile container (e.g., a syringe) and, optionally, may be lyophilized and reconstituted (e.g., with sterile PBS) prior to administration.

A single dose of heat shock protein-encoding nucleic acid molecule in a non-targeting carrier to administer to an animal to treat a disease is from about 0.1 µg to about 200 µg of total recombinant molecules per kilogram (kg) of body weight, e.g. from about 0.5 µg to about 150 µg of total recombinant molecules per kg of body weight, or from about 1 µg to about 10 µg of total recombinant molecules per kg of body weight.

In another embodiment, the composition is formulated for intramuscular administration, or by other convenient methods known in the art, e.g. parenteral, oral, topical or transdermal administration. In another embodiment said administration is performed in a manner compatible with uptake of said construct by intradermal dendritic cells (DC) or other tissue-residing antigen presenting cells (APC), e.g. intramuscular.

### Subjects and Therapeutic Use

According to certain embodiments, the compositions, methods, kits and medicaments of the invention are used for treating or preventing dysbiosis in a subject in need thereof. In other embodiments, the compositions, methods, kits and medicaments of the invention are used for promoting the growth of beneficial microbiota in the GI tract. In other embodiments, the compositions, methods, kits and medicaments of the invention are used for inhibiting the growth of detrimental microbiota in the GI tract. In other embodiments, the compositions, methods, kits and medicaments of the invention are used for modifying (e.g. enhancing) microbiota biodiversity in the GI tract. Each possibility represents a separate embodiment of the invention. In other embodiments, the compositions, methods, kits and medicaments of the invention are used for treating or preventing the appearance of symptoms of dysbiosis in a subject in need thereof. In some embodiments, symptoms of dysbiosis may include but are not limited to, abdominal distension, regular/frequent episodes of diarrhea, frequent stools, recent onset/chronic diarrhea or diarrhea for 1-3 months, poor tolerance/intolerance of sugars, flatulence, rotten egg burps, and meal-related bloating. In some embodiments, these GI symptoms may be accompanied by non-GI symptoms such as constant fatigue and “brain fog”. Each possibility represents a separate embodiment of the invention.

In another embodiment, the compositions and methods of the invention are useful for preventing or reversing a transition of GI microflora from a profile characterized by primarily obligate anaerobic bacteria (e.g. about 97% and/or exceeding by two or three orders of magnitude the abundance of facultative anaerobic and aerobic bacteria) to a profile characterized by enhanced abundance of aerobic and/or facultative anaerobic bacteria. In another embodiment the dysbiosis is chronic. In another embodiment said dysbiosis is acute.

In another embodiment, the subject is characterized by GI expansion of at least one of Bacteroidaceae, Enterobacteriaceae and Enterococcaceae (e.g. compared to a healthy control subject, or to the subject at a previous time point). In yet another embodiment, said subject is characterized by GI expansion of at least one of Bacteroidaceae, Enterobacteriaceae, Enterococcaceae and Porphyromonadaceae. In another embodiment, said subject is characterized by GI expansion of facultative anaerobic Proteobacteria.

In another embodiment, treatment comprises inhibiting the growth of detrimental microbiota in the GI tract. In another embodiment, the detrimental microbiota comprise at least one pathogen species belonging to the Bacteroidaceae, Enterobacteriaceae and/or Enterococcaceae family. In another embodiment, the detrimental microbiota comprise at least one pathogen species belonging to the Bacteroidaceae, Enterobacteriaceae, Enterococcaceae and/or Porphyromonadaceae family. In another embodiment, said detrimental microbiota comprise at least one Enterococcus, Escherichia or Bacteroides species. In another embodiment, said detrimental microbiota comprise at least one Enterococcus, Escherichia, Bacteroides or Parabacteroides species. In another embodiment, said detrimental microbiota comprise at least one Enterococcus species, at least one Escherichia species and at least one Bacteroides species. In another embodiment, said detrimental microbiota comprise at least one Enterococcus species, at least one Escherichia species, at least one Bacteroides species and at least one Parabacteroides species.

In another embodiment, treatment comprises promoting the growth of beneficial microbiota in the GI tract. In another embodiment the beneficial microbiota comprise at least one Firmicutes or Bifidobacteriaceae species. In another embodiment, the beneficial microbiota comprise at least one Firmicutes species and at least one Bifidobacteriaceae species. In another embodiment, said beneficial bacteria comprise at least one Lachnospiraceae, Lactobacillus, Clostridiales, Coprococcus, Ruminococcus and/or Turicibacter species. In another embodiment the method is used for modifying microbiota biodiversity in the GI tract. In another embodiment, treatment comprises retaining or restoring the dominance of obligate anaerobic GI bacteria.

In another embodiment of the methods of the invention the subject is afflicted with a disease or disorder associated with a bacterial, viral or parasitic infection or overgrowth. Each possibility represents a separate embodiment of the invention. In another embodiment, the disease or disorder is associated with infection by drug-resistant bacteria. In another embodiment the drug-resistant bacteria are selected from the group consisting of Enterococcus, Escherichia and Bacteroides species. In another embodiment, said drug-resistant bacteria are selected from the group consisting of detrimental bacteria as disclosed herein, e.g. Proteobacteria, Gammaproteobacteria, Enterobacteriales, Enterobacteriaceae and/or Escherichia species. In another embodiment, said disease or disorder is a non-autoimmune inflammatory disorder of the GI tract.

As used herein, the term “infection” refers to the invasion and multiplication of pathogenic microorganisms that are not normally present within the body. An “overgrowth” relates to excessive proliferation of an organism that is normally present in the body, which may induce or enhance pathological processes due to e.g. activation of the host’s immune response. In another embodiment of the methods of the invention, the subject is human. In another embodiment said subject exhibits dysbiosis. In another embodiment said subject is at risk for developing dysbiosis, e.g. due to exposure to antibiotics, immunosuppressants, chemotherapy or other treatments, conditions or procedures (e.g. surgery) associated with an enhanced risk for developing dysbiosis. In another embodiment said subject is under a treatment regimen with at least one of antimicrobial agents, parenteral nutrition, or immune suppressive agents. In another embodiment said subject is hospitalized, for example patients at intensive care units (e.g. due to acute cerebral infarction, severe burn damage or other critical care patients).

Thus, in another embodiment, the methods of the invention are used for treating or preventing hospital-acquired infections. Hospital-acquired infections, also known as healthcare-associated infections (HAI), are nosocomially acquired infections that are typically not present or might be incubating at the time of admission. These infections are usually acquired after hospitalization and manifest > 48 hours after admission to the hospital. These infections commonly include pneumonia and other respiratory infections (e.g. ventilator-associated pneumonia), urinary tract infections (e.g. catheter-associated infections), surgical site infections, clinical sepsis (e.g. central line-associated bloodstream infections), and gastrointestinal infections.

In another embodiment, said dysbiosis is associated with gut barrier dysfunction. In another embodiment, said dysbiosis is associated with a GI disorder selected from the group consisting of: irritable bowel syndrome (IBS), celiac disease, small intestinal bacterial overgrowth (SIBO), leaky gut syndrome, and diverticular disease. In another embodiment said dysbiosis is associated with a GI disorder selected from the group consisting of: IBS, celiac disease, SIBO, leaky gut syndrome, diverticular disease, and AIDS enteropathy. In another embodiment said dysbiosis is associated with a GI disorder selected from the group consisting of: IBS, SIBO, leaky gut syndrome, and diverticular disease. Each possibility represents a separate embodiment of the invention. In other embodiments the dysbiosis is associated with e.g. a chronic lung disease, obesity, hypertension or metabolic diseases associated with expansion of proteobacteria.

Irritable bowel syndrome (IBS) is a common disorder that has a pronounced effect on the quality of life and that accounts for a large proportion of healthcare costs. The disorder is characterized by lower abdominal pain, bloating, diarrhea, constipation, or constipation alternating with diarrhea. Altered bowel motility, visceral hyperalgesia, food allergy, bacterial overgrowth, psychosomatic factors, stress associated with the myenteric nervous system have all been proposed as playing a part in the pathogenesis of IBS. Gastrointestinal inflammation may also be associated with irritable bowel syndrome, along with stress.

Celiac disease is an autoimmune disease manifested in genetically susceptible people caused by intolerance to gluten, resulting in mucosal inflammation and villous atrophy, which causes malabsorption. Symptoms usually include diarrhea and abdominal discomfort. Diagnosis is by small-bowel biopsies showing characteristic though not specific pathologic changes of villous atrophy that resolve with a strict gluten-free diet.

Small intestinal bacterial overgrowth (SIBO), also termed small bowel bacterial overgrowth syndrome, is a disorder of excessive bacterial growth in the small intestine (bacterial counts of >10⁵/mL). SIBO can result from alterations in intestinal anatomy (e.g. due to surgery or partial obstruction) or GI motility, or from lack of gastric acid secretion. This condition can lead to vitamin deficiencies, fat malabsorption, and undernutrition. The most frequent symptoms are abdominal discomfort, diarrhea, bloating, and excess flatulence.

Increased intestinal permeability (abnormally excessive opening of intercellular tight junctions) allows passage of microbes, microbial products, and foreign antigens into the mucosa and bloodstream, with subsequent possible development of immune and/or inflammatory reactions. Such reactions, collectively referred to as “leaky gut syndrome”, may result in chronic inflammation and be associated with the development of additional GI pathologies such as celiac disease and IBS.

Diverticular disease includes diverticulosis and diverticulitis, which are related digestive conditions that affect the large intestine. The disease is characterized by the development of small bulges or pockets termed diverticula in the lining of the intestine. Asymptomatic diverticular disease is known as diverticulosis, while diverticular disease associated with an infection and/or inflammation is known as diverticulitis. In a particular embodiment, the subject is afflicted with colonic diverticulitis.

In another embodiment, the subject is afflicted with a disease or disorder associated with a viral infection, e.g. HIV. In a particular embodiment the subject is afflicted with AIDS enteropathy. AIDS enteropathy (also known as idiopathic AIDS enteropathy or HIV enteropathy), which is characteristic of a subset of patients with advanced human immunodeficiency virus (HIV), is defined as a reduction in small bowel villous surface area associated with chronic diarrhea without pathogen infection, due to intestinal mucous damage by HIV infection.

In other embodiments, the compositions, methods, kits and medicaments of the invention comprise, or are used in combination with, at least one antibiotic, probiotic or prebiotic agent. Exemplary antibiotic agents include, but are not limited to, nitroimidazole, macrolide, and betalactam antibiotics. Typically, when used in connection with antibiotic agents, the plasmid may conveniently be administered towards the end of the treatment regimen with the antibiotic agent, or during the recovery phase (after antibiotic administration has been completed or terminated). Exemplary prebiotic agents include, but are not limited to, lactulose, lignin, cellulose, hemicelluloses, β-glucans, pectin, gums, resistant starch, dextrin, psyllium, inulin, fructooligosaccharides, and polydextrose. Exemplary probiotic agents include, but are not limited to, various beneficial lactobacillus species as disclosed herein, or a fecal microbiota transplant (FMT). In other embodiments, the compositions, methods, kits and medicaments of the invention are used in combination with a specialized diet, including, but not limited to, enteral or parenteral liquid formulations, reported to be associated with the development of dysbiosis, or a diet prescribed for the management of dysbiosis. According to exemplary embodiments, the compositions, methods, kits and medicaments of the invention are used in combination with fiber-enriched, fructose-reduced, elemental, total liquid enteral, SIBO-specific, total parenteral, and peripheral parenteral diet. Liquid enteral diet refers to the introduction of a nutritionally complete liquid formula directly into the stomach or small intestine via a designated (e.g. nasogastric) tube. Parenteral diet (parenteral nutrition) refers to the delivery of liquid nutrition into a vein. When the diet is used as the exclusive source of nutrition, it is referred to as total liquid enteral diet, or total parenteral diet, respectively. Elemental diet comprises liquid nutrients in an easily assimilated form (for example, nitrogen is provided in the form of free amino acids rather than as whole or partial protein). It is usually composed of amino acids, fats, sugars, vitamins, and minerals. Elemental diet may be administered orally or by use of a gastric feeding tube or intravenous feeding. Fiber enriched diet typically refers to specialized fiber-enriched enteral formulations (e.g. Nutrison multifibre, containing 1.5 g/100 ml of soluble and non-soluble fibers at a 1:1 ratio), but may also refer in some embodiments to prescribed oral diets including daily ingestion of e.g. 30 gr of fiber or more. Fructose-reduced diets may be e.g. low-fermentable oligo-saccharides, disaccharides, monosaccharides, and polyol (FODMAP) diet, or enteral nutrition formulations devoid of added fructose.

The subject to be treated by the compositions, methods pharmaceutical packs and medicaments of the invention, also referred to herein as a subject in need thereof, is a mammalian and preferably a human subject. In certain embodiments, the subject has been diagnosed as suffering from dysbiosis. In certain other embodiments, the subject is determined by the skilled artisan to be at risk for developing dysbiosis. Each possibility represents a separate embodiment of the invention.

Various methods for diagnosing dysbiosis are known in the art, and include without limitation, breath-testing methods, small-bowel culture techniques and culture-independent techniques such as high-throughput next-generation sequencing. For example, the Comprehensive Digestive Stool Analysis (CDSA, a non-invasive evaluation of gastrointestinal function that includes analyses of digestion, absorption, bacterial balance, yeast and parasites), or the GA-map Dysbiosis Test (Genetic Analysis AS, Oslo, Norway, based on DNA profiling using probes targeting variable regions of the bacterial 16S rRNA gene), may be used.

In various embodiments, a subject at risk of developing dysbiosis is under a treatment regimen with at least one agent selected from the group consisting of anti-microbial agents, parenteral nutrition, or immune suppressive agents, wherein each possibility represents a separate embodiment of the invention. Immune suppressive agents are drugs that inhibit or prevent activity of the immune system, including, but not limited to glucocorticoids, cytostatics, antibodies and drugs acting on immunophilins.

In particular embodiments, the composition is for administering to an individual who is not diagnosed with a T-cell mediated inflammatory autoimmune disease such as for example, rheumatoid arthritis, collagen II arthritis, multiple sclerosis, autoimmune neuritis, systemic lupus erythematosus, psoriasis, Sjogren’s disease, thyroid disease, sarcoidosis, autoimmune uveitis, inflammatory bowel disease (Crohn’s and ulcerative colitis) or autoimmune hepatitis. In a particular embodiment, the composition is for administering to an individual who is not diagnosed with type 1 diabetes. In a particular embodiment, the composition is for administering to an individual who is not diagnosed with an angiogenesis related disease. In another particular embodiment, said subject is not HLA-B27 positive. In another embodiment, the subject is not diagnosed with a Th1-mediated condition.

As disclosed herein, embodiments of the invention define and disclose the treatment of new patient populations. In some embodiments, the methods of the invention comprise determining whether the subject is amenable for treatment by a nucleic acid construct as disclosed herein, by determining the levels of certain bacterial taxa, disclosed herein to be specifically modulated by the constructs of the invention. To this end, a gut microbiota sample of the subject is provided, and the relative abundance of said taxa is evaluated and compared to their healthy control levels.

In some embodiments, the methods of the invention comprise obtaining a sample comprising GI bacteria from the subject. Conveniently, the sample is a fecal sample. In other embodiments, samples may be collected from different parts of the GI tract (or secretions or fluids thereof), e.g. from mucosal tissue (e.g. gut mucosa or oral mucosa), or saliva. In additional embodiments, the methods comprise determining the relative abundance of at least one bacterial strain in the sample, in particular at least one of the detrimental bacterial taxa as disclosed herein. In some embodiments, the methods comprise determining the relative abundance of at least one of Bacteroidaceae, Enterobacteriaceae and Enterococcaceae in the sample. In other embodiments, the methods comprise determining the relative abundance in the sample of at least one Enterococcus, Escherichia or Bacteroides species.

For example, methods for evaluating the abundance of microorganisms are available and include, without limitation, sequencing, amplification and/or other molecular procedures known in art. In another embodiment, the evaluation may comprise sequencing of a reporter gene or sequence, e.g. rRNA. In other embodiments, the evaluation comprises sequencing of other single-copy genes or genomic areas used for taxonomic identification. In a particular embodiment, the reporter gene or sequence is a 16S rRNA. In another embodiment, the reporter gene or sequence is an intergenic spacer region located between rRNA genes, and the evaluation involves rRNA intergenic spacer analysis (RISA). In another embodiment, relative abundance or diversity may be assessed using meta-genomic sequencing. In another embodiment, relative abundance or diversity may be assessed using genomic sequencing of microorganisms. In another embodiment, a bioinformatic analysis can be used to identify and optionally quantify the abundance of the various populations present in the sample, e.g. using learning and pattern recognition algorithms.

In other embodiments, the methods include comparing the abundance values measured to those corresponding to a healthy control. In additional embodiments, significantly enhanced values (statistically significant enhancement in the abundance of a detrimental bacterium as disclosed herein) compared to the control sample indicate that said subject is amenable for treatment. In various embodiments, the control may be e.g. a sample from at least one healthy individual, a panel of control samples from a set of healthy individuals, and a stored set of data obtained from healthy individuals. Each possibility represents a separate embodiment of the invention.

In other embodiments, the methods further comprise, administering to said subject, if determined amenable for treatment, a nucleic acid construct comprising a nucleic acid sequence encoding a mammalian HSP90, or an active fragment thereof, wherein the nucleic acid sequence is operatively linked to one or more transcription control sequences.

“Operational taxonomic unit (OTU, plural OTUs)” refers to a terminal leaf in a phylogenetic tree and is defined by a specific genetic sequence and all sequences that share sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S sequence or a portion of the 16S sequence or it may be a functionally conserved housekeeping gene found broadly across the eubacterial kingdom. OTUs share at least 95%, 96%, 97%, 98%, or 99% sequence identity. OTUs are frequently defined by comparing sequences between organisms. Sequences with less than 95% sequence identity are not considered to form part of the same OTU.

In microbiology, “16S sequencing” or “16S rRNA” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi.

In various embodiments as disclosed herein, the invention relates to a nucleic acid construct comprising a nucleic acid sequence encoding a mammalian HSP90 (in particular HSP90 alpha, e.g. isoform 2), or an active fragment thereof, wherein the nucleic acid sequence is operatively linked to one or more transcription control sequences, for use in treating or preventing dysbiosis in a subject in need thereof.

In various embodiments as disclosed herein, the invention relates to a nucleic acid construct comprising a nucleic acid sequence encoding a mammalian HSP90 (in particular HSP90 alpha, e.g. isoform 2), or an active fragment thereof, wherein the nucleic acid sequence is operatively linked to one or more transcription control sequences, for use in treating or preventing gut barrier dysfunction in a subject in need thereof.

In yet other embodiments as disclosed herein, the invention relates to a nucleic acid construct comprising a nucleic acid sequence encoding a mammalian HSP90 (in particular HSP90 alpha, e.g. isoform 2), or an active fragment thereof, wherein the nucleic acid sequence is operatively linked to one or more transcription control sequences, for use in treating an inflammatory GI disorder in a subject in need thereof, wherein the use comprises determining if said subject is amenable for treatment by:
a) obtaining a sample comprising GI bacteria from the subject (in particular a fecal sample);
b) determining the relative abundance of at least one of Bacteroidaceae, Enterobacteriaceae and Enterococcaceae in the sample; and
c) comparing the abundance values measured to those corresponding to a healthy control, wherein significantly enhanced values compared to the control values indicate that said subject is amenable for the treatment.

In another embodiment, the use further comprises administering to said subject, if determined amenable for said treatment, a nucleic acid construct comprising a nucleic acid sequence encoding a mammalian HSP90 or an active fragment thereof (in particular HSP90 alpha, e.g. isoform 2), wherein the nucleic acid sequence is operatively linked to one or more transcription control sequences.

### Treatment-Resistant Conditions and Combination Therapy

Traditional therapies for inflammatory autoimmune diseases have relied on immunosuppressive medications that globally dampen immune responses. These agents are highly effective for many patients and thus remain the current first line of treatment or “gold standard” of care. However, long-term treatments with high doses are often needed to maintain disease control, leaving the patient susceptible to life-threatening opportunistic infections and long-term risk of malignancy. In addition, the benefits of many of these drugs are counterbalanced by toxicity and serious side effect profiles. Thus, there has been a push for the development of more specific strategies that lower the risk of systemic immune suppression and improve tolerability.

While several immunomodulatory treatments, including tumor necrosis factor (TNF) inhibitors and other anti-cytokine antibodies such as the (IL)-12/IL-23 inhibitor ustekinumab, have already been approved for gastroenterological, rheumatological and dermatological inflammatory conditions, about 30%-40% of patients treated with these drugs do not respond to treatment (primary non-response) and 23-46% among those responding lose response over time (secondary non-response), suggesting the need for additional therapeutic modalities.

In some embodiments, the subject to be treated by the compositions and methods of the invention is afflicted with a disease or condition resistant to an immunomodulatory treatment selected from immune suppressive treatment and anti-cytokine immunomodulatory treatment (or is otherwise not amenable for said treatment, e.g. due to toxicity or side effects). For example, without limitation, the subject may be resistant to immune suppressive treatments such as glucocorticoids (e.g. prednisone, dexamethasone, and hydrocortisone) and cytostatics (including antimetabolites e.g. methotrexate, azathioprine), and/or to anti-cytokine immunomodulatory treatment such as tumor necrosis factor alpha (TNF-α) antagonists or inhibitors. According to additional non-limitative examples, immunomodulatory treatment approved for the treatment of autoimmune diseases include antibody-based drugs directed to cytokine targets or other immune targets including, but not limited to, TNF (e.g. adalimumab, certolizumab, certolizumab pegol, golimumab, infliximab, etanercept), IL6R (e.g. tocilizumab, sarilumab), IL12/ IL23 (e.g. ustekinumab), integrin receptor (e.g. vedolizumab), IL17RA (brodalumab) and IL17A (ixekizumab). Each possibility represents a separate embodiment of the invention.

In other embodiments, an HSP90-encoding construct as disclosed herein may advantageously be administered in concurrent or sequential combination with an anti-cytokine immunomodulatory treatment (including, but not limited to, anti-cytokine antibody-based agents and non-antibody cytokine receptor blockers). In another embodiment, said anti-cytokine immunomodulatory treatment is a TNF-α antagonist or inhibitor. In yet another embodiment, said HSP90-encoding construct is not administered in combination with an anti-cytokine immunomodulatory treatment. In a further embodiment, said HSP90-encoding construct is administered as the sole active ingredient. Each possibility represents a separate embodiment of the invention.

In other embodiments, the invention relates to a therapeutic combination of (i) an anti-cytokine immunomodulatory treatment such as a TNF-α antagonist or inhibitor and (ii) a nucleic acid construct comprising a nucleic acid sequence encoding a mammalian HSP90, or an active fragment thereof, wherein the nucleic acid sequence is operatively linked to one or more transcription control sequences. In various embodiments, the active ingredients of the combination may be co-formulated in the form of a pharmaceutical composition, or be provided in the form of a kit or pharmaceutical pack comprising separate pharmaceutical compositions, each comprising an active ingredient.

In some embodiments, combinations and methods as disclosed herein may be used for treating or preventing a pathology associated with gastrointestinal dysbiosis in a subject in need thereof and/or treating or alleviating the symptoms of a treatment-resistant inflammatory disorder.

As used herein, the term “treatment resistant” encompasses both primary and secondary non-responders as disclosed herein. Further, in some embodiments, a treatment-resistant inflammatory disorder in a subject may be manifested as lack of adequate therapeutic response to two or more immunomodulatory drugs or drug categories (for example, immune suppressive treatment and anti-cytokine immunomodulatory treatment as disclosed herein).

In another exemplary embodiment, the compositions, methods and kits of the invention may be used for maintenance therapy, to prevent the development of dysbiosis or GI symptoms in a subject in which immunomodulatory treatment attenuation or replacement is indicated. For example, in a subject under treatment regimen with an antibody-based drug, efficacy may deteriorate over time (e.g. due to development of host antibodies to the drug), or safety concerns (e.g. opportunistic infections due to excessive immune suppression or other side effects) may develop over time, prompting withdrawal of the drug and consideration of alternative treatment regimens. An HSP90-encoding construct in accordance with the invention may be administered to the subject during the period of treatment replacement, concomitantly with the existing and/or replacement drug, in order to maintain GI homeostasis and gut barrier function.

In another embodiment, the invention relates to a method of treating or alleviating the symptoms of a treatment-resistant inflammatory disorder, comprising administering to the subject, in concurrent or sequential combination, (i) a nucleic acid construct comprising a nucleic acid sequence encoding a mammalian HSP90, or an active fragment thereof, wherein the nucleic acid sequence is operatively linked to one or more transcription control sequences, and (ii) a TNF-α antagonist or inhibitor. In another embodiment the disorder is resistant to an anti-cytokine immunomodulatory treatment. In another embodiment the anti-cytokine immunomodulatory treatment is a TNF-α antagonist or inhibitor. In another embodiment the disorder is resistant to an immune suppressive treatment. In another embodiment the immune suppressive treatment is selected from the group consisting of glucocorticoids and cytostatics. In another embodiment the disorder is an autoimmune disease. In another embodiment the disorder is selected from the group consisting of inflammatory bowel disease (IBD) and rheumatoid arthritis. In another embodiment the disorder is a chronic inflammatory disease. In another embodiment said disorder is a non-autoimmune inflammatory disease. In another embodiment, the subject is not concomitantly afflicted with an inflammatory autoimmune disease. In another embodiment the TNF-α antagonist or inhibitor is selected from the group consisting of anti-TNF antibody-based agents such as adalimumab (Humira), certolizumab, certolizumab pegol (Cimzia), golimumab (Simponi), and infliximab (Remicade), as well as non-antibody cytokine receptor blockers such as etanercept (Enbrel, which is a fusion protein combining two naturally occurring soluble human 75-kilodalton TNF receptors linked to an Fc portion of an IgG1). It is to be understood, that the use of drugs approved or recognized as biosimilars of a specific TNF-α antagonist or inhibitor disclosed herein is further contemplated.

In another embodiment, the invention relates to a method of treating or preventing a pathology associated with gastrointestinal dysbiosis in a subject in need thereof, comprising administering to the subject, in concurrent or sequential combination (i) a nucleic acid construct comprising a nucleic acid sequence encoding a mammalian HSP90, or an active fragment thereof, wherein the nucleic acid sequence is operatively linked to one or more transcription control sequences, and (ii) a TNF-α antagonist or inhibitor.

In some embodiments, the method of treating or preventing a pathology associated with gastrointestinal dysbiosis or of treating or alleviating the symptoms of a treatment-resistant inflammatory disorder further comprises determining said subject as amenable for treatment as disclosed herein. In another embodiment, the method further comprises:
a) obtaining a sample comprising GI bacteria from the subject;
b) determining the relative abundance of at least one of Bacteroidaceae, Enterobacteriaceae and Enterococcaceae in the sample;
c) comparing the abundance values measured to those corresponding to a healthy control, wherein significantly enhanced values compared to the control values indicate that said subject is amenable for treatment.

The following examples are presented in order to more fully illustrate some embodiments of the invention. They should, in no way be construed, however, as limiting the broad scope of the invention.

### EXAMPLES

### Methods

### Animal Model

A model of intestinal damage induced by TNBS (2,4,6-trinitrobenzenesulfonic acid) in C57BL/6 male mice was used in this study. For induction of GI pathology, C57BL/6 mice (age: > 8 weeks) were anesthetized for 90-120 minutes and received an intrarectal administration of TNBS (40 µl at 150 mg/kg) dissolved in a 1:1 mixture of 0.9% NaCl and 100% ethanol. Mice were further administered with DNA constructs as detailed below.

Control C57BL/6 (naïve) male mice were administered in the same technique with a saline solution, and used to evaluate the effect of the test constructs in healthy control animals. All mice were fasted overnight prior to induction of intestinal damage by TNBS.

### DNA Plasmid and Dosage Form Preparation

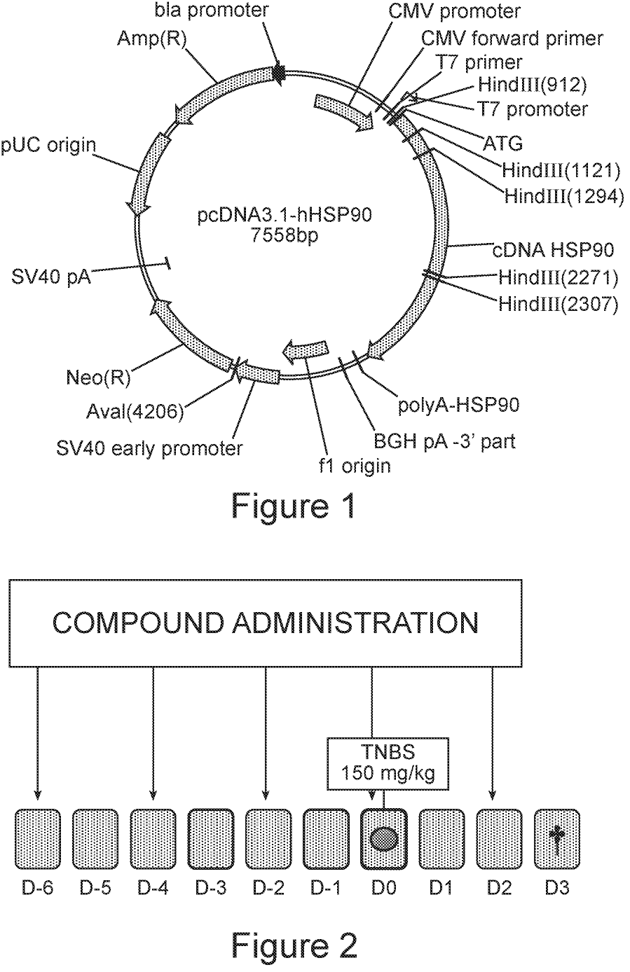

A recombinant construct encoding human HSP90 (alpha isoform 2) under CMV promoter expression control was constructed based on the pCDNA3.1 expression vector (FIG. 1, hereinafter “HSP90 plasmid”). The empty vector pcDNA3.1 was used as a control construct.

The backbone vector pcDNA3.1 (+) further included an ampicillin resistance cassette (Amp(R)). For the cloning, Homo sapiens heat shock protein 90 alpha family class A member 1 (HSP90AA1), transcript variant 2, cDNA (NM_005348.3) was used, as follows:

gcatgcgtag gcgcgcggcc gcggcggcgg ctggggaggg ttcttccgga aggttcggga
ggcttctgga aaaagcgccg cgcgctgggc gggcccgtcg ctatataagg caggcgcggg
ggtggcgcgt cagttgcttc agcgtcccgg tgtggctgtg ccgttggtcc tgtgcggtca
cttagccaag atgcctgagg aaacccagac ccaagaccaa ccgatggagg aggaggaggt
tgagacgttc gcctttcagg cagaaattgc ccagttgatg tcattgatca tcaatacttt
ctactcgaac aaagagatct ttctgagaga gctcatttca aattcatcag atgcattgga
caaaatccgg tatgaaagct tgacagatcc cagtaaatta gactctggga aagagctgca
tattaacctt ataccgaaca aacaagatcg aactctcact attgtggata ctggaattgg
aatgaccaag gctgacttga tcaataacct tggtactatc gccaagtctg ggaccaaagc
gttcatggaa gctttgcagg ctggtgcaga tatctctatg attggccagt tcggtgttgg
tttttattct gcttatttgg ttgctgagaa agtaactgtg atcaccaaac ataacgatga
tgagcagtac gcttgggagt cctcagcagg gggatcattc acagtgagga cagacacagg
tgaacctatg ggtcgtggaa caaaagttat cctacacctg aaagaagacc aaactgagta
cttggaggaa cgaagaataa aggagattgt gaagaaacat tctcagttta ttggatatcc
cattactctt tttgtggaga aggaacgtga taaagaagta agcgatgatg aggctgaaga
aaaggaagac aaagaagaag aaaaagaaaa agaagagaaa gagtcggaag acaaacctga
aattgaagat gttggttctg atgaggaaga agaaaagaag gatggtgaca agaagaagaa
gaagaagatt aaggaaaagt acatcgatca agaagagctc aacaaaacaa agcccatctg
gaccagaaat cccgacgata ttactaatga ggagtacgga gaattctata agagcttgac
caatgactgg gaagatcact tggcagtgaa gcatttttca gttgaaggac agttggaatt
cagagccctt ctatttgtcc cacgacgtgc tccttttgat ctgtttgaaa acagaaagaa
aaagaacaac atcaaattgt atgtacgcag agttttcatc atggataact gtgaggagct
aatccctgaa tatctgaact tcattagagg ggtggtagac tcggaggatc tccctctaaa
catatcccgt gagatgttgc aacaaagcaa aattttgaaa gttatcagga agaatttggt
caaaaaatgc ttagaactct ttactgaact ggcggaagat aaagagaact acaagaaatt
ctatgagcag ttctctaaaa acataaagct tggaatacac gaagactctc aaaatcggaa
gaagctttca gagctgttaa ggtactacac atctgcctct ggtgatgaga tggtttctct
caaggactac tgcaccagaa tgaaggagaa ccagaaacat atctattata tcacaggtga
gaccaaggac caggtagcta actcagcctt tgtggaacgt cttcggaaac atggcttaga
agtgatctat atgattgagc ccattgatga gtactgtgtc caacagctga aggaatttga
ggggaagact ttagtgtcag tcaccaaaga aggcctggaa cttccagagg atgaagaaga
gaaaaagaag caggaagaga aaaaaacaaa gtttgagaac ctctgcaaaa tcatgaaaga
catattggag aaaaaagttg aaaaggtggt tgtgtcaaac cgattggtga catctccatg
ctgtattgtc acaagcacat atggctggac agcaaacatg gagagaatca tgaaagctca
agccctaaga gacaactcaa caatgggtta catggcagca aagaaacacc tggagataaa
ccctgaccat tccattattg agaccttaag gcaaaaggca gaggctgata agaacgacaa
gtctgtgaag gatctggtca tcttgcttta tgaaactgcg ctcctgtctt ctggcttcag
tctggaagat ccccagacac atgctaacag gatctacagg atgatcaaac ttggtctggg
tattgatgaa gatgacccta ctgctgatga taccagtgct gctgtaactg aagaaatgcc
accccttgaa ggagatgacg acacatcacg catggaagaa gtagactaat ctctggctga
gggatgactt acctgttcag tactctacaa ttcctctgat aatatatttt caaggatgtt
tttctttatt tttgttaata ttaaaaagtc tgtatggcat gacaactact ttaaggggaa
gataagattt ctgtctacta agtgatgctg tgatacctta ggcactaaag cagagctagt
aatgcttttt gagtttcatg ttggtttatt ttcacagatt ggggtaacgt gcactgtaag
acgtatgtaa catgatgtta actttgtggt ctaaagtgtt tagctgtcaa gccggatgcc
taagtagacc aaatcttgtt attgaagtgt tctgagctgt atcttgatgt ttagaaaagt
attcgttaca tcttgtagga tctacttttt gaacttttca ttccctgtag ttgacaattc
tgcatgtact agtcctctag aaataggtta aactgaagca acttgatgga aggatctctc
cacagggctt gttttccaaa gaaaagtatt gtttggagga gcaaagttaa aagcctacct
aagcatatcg taaagctgtt caaaaataac tcagacccag tcttgtggat ggaaatgtag
tgctcgagtc acattctgct taaagttgta acaaatacag atgagttaaa agatattgtg
tgacagtgtc ttatttaggg ggaaagggga gtatctggat gacagttagt gccaaaatgt
aaaacatgag gcgctagcag gagatggtta aacactagct gctccaaggg ttgacatggt
cttcccagca tgtactcagc aggtgtgggg tggagcacac gtaggcacag aaaacaggaa
tgcagacaac atgcatcccc tgcgtccatg agttacatgt gttctcttag tgtccacgtt
gttttgatgt tattcatgga ataccttctg tgttaaatac agtcacttaa ttccttggcc
ttaaaa (SEQ ID NO: 2).

For small scale plasmid DNA production, DG1 chemically competent E. coli cells were transformed with the plasmid preparation. Amplification was performed in 5 ml of LB medium + 100 µg/ml Ampicillin at 37° C. DNA extraction was performed by Invisorb Spin DNA extraction kit (miniprep).

Plasmid DNA was prepared by diluting in DNAse/RNAse free PBS buffer pH 7.5 to a concentration of 6 mg/ml and kept at 4° C. during the whole treatment period. Sampling of the solution were made in aseptic conditions using sterile and pyrogen-free material.

The day before the first dosing, plasmid solutions were diluted in DNAse/RNAse free PBS buffer pH 7.5 according to Table 2.

TABLE 2

Dilution and injection doses of empty vector and HSP90 plasmid

| Test item | Initial concentration (mg/ml) | Dilution in buffer | Volume of injection (µL) | Injected dose (mg/kg) | Injected dose per mouse^{∗} (mg/animal) |
|---|---|---|---|---|---|
| HSP90 plasmid | 6.0 | 48 | 100 | 0.5 | 0.0125 |
| Empty vector | 4.6 | 37 | 100 | 0.5 | 0.0125 |

^{∗} For a mouse weighting 25 gr. The dose was adjusted to each animal according to the recorded body weight.

### Experimental Groups

The study included 40 C57BL/6 mice that were allocated to the following five treatment groups using a manual randomization procedure:
Group #1: naïve (no treatment) (CTL; n=5)
Group #2: HSP90 plasmid (CTL_HSP90; n=5)
Group #3: TNBS + saline buffer (TNBS_PBS; n=10)
Group #4: TNBS + empty plasmid (TNBS_emptyPlasm; n=10)
Group #5: TNBS + HSP90 plasmid (TNBS_HSP90; n=10)

Administration of control construct or HSP90 plasmid (both in a dose of 0.5 mg/kg), as well as the saline buffer control, was performed by an intramuscular route. The dosage forms were injected into the thighs, alternating between each of the posterior legs of the animal. Administration began 6 days before TNBS administration, and then - every two days up to and including day 2 post TNBS administration, namely at days -6, -4, -2, 0, and +2, wherein day 0 was defined as the day of TNBS intrarectal administration (marked as D-6, D-4, D-2, D0 and D2, respectively, in FIG. 2, illustrating the experimental setting).

### Feces Sampling and Microbiome Analysis

Feces were collected (150-200 mg per sample) for each mouse in all treatment groups at three time points, i.e., on day -7 (one day before the first plasmid injection), on day -1 (one day before intra-rectal administration of TNBS) and on day +3 (at sacrifice).

The fecal microbiome was analyzed by amplicon analysis of the 16S ribosomal RNA (rRNA) gene which is a common sequencing approach to analyze the microbiome. In this method, a 16S rRNA region is amplified by PCR with primers that recognize highly conserved regions of the gene and sequenced. More specifically, gene specific primers for the bacterial 16S rRNA were used to amplify the V3-V4 region. Primers were based on the Illumina’s dual indexing sequencing principles, each gene-specific primer was flanked with an index sequence used to identify the corresponding sample along with the Illumina adapters that were complementary to those found on the flow cell. Amplified PCR products were purified and normalized using the SequalPrep Normalization Plate Kit (Life technologies, CA, USA).

Library size was controlled using the Agilent TapeStation HS1000 Screen Tape (Agilent technologies, USA) and final concentrations were determined by using a SYBR green quantitative PCR (qPCR) assay with primers specific to the Illumina adapters (Kapa Q-PCR Universal Library Quantification, Kapa Biosystems, Wilmington, Massachusetts, USA). Libraries were denatured and diluted at 12 pM before being mixed with 5% of Illumina PhiX control libraries. Mixed Phix/16S libraries were sequenced in multiplex on the MiSeq machine with the MiSeq v3 chemistry to perform paired-end 300bp sequencing. During sequencing the MiSeq was running Real Time Analysis software (RTA) version 1.18.54 and 2.5.05 MiSeq Control software (MCS). Sequence demultiplexing was performed automatically by MiSeq Reporter software (MRS) version 2.5.

Based on this 16S rRNA sequence analysis, the proportion of taxa present in the fecal samples at each taxonomic level (phylum, family and genus) was determined.

An operational taxonomic unit (OTU) is an operational definition used to classify groups of closely related individuals. OTU clustering was performed (QIIME, open-ref, uclust, RDP, GreenGenes). Raw data was preprocessed (QIIME, prinseq, usearch) before being used for OTU analysis. First amplicons were built by assembling the paired-end reads. Low quality score bases were trimmed and low average quality score sequences were filtered. Finally, the chimera predicted sequences were filtered before using the sequence data for OTU analysis. Annotated OTU table was used for sample description via composition summary plots, alpha- and beta- diversity analysis as well as differential feature statistical analysis.

### Comparative Statistical Analysis

Statistical analysis of 16S data was performed using DESeq2 official extension within phyloseq R package. The DESeq2-phyloseq approach has the advantage of using the same statistical framework as for RNA-Seq data, increasing the statistical power and allowing for more complex experiment designs.

### Example 1. The Effect of HSP90 Plasmid on Gastrointestinal (GI) Microbiota of Healthy Mice

The effect of HSP90 plasmid administration on fecal microbiota over time was examined in healthy mice (Group #2), by differential abundance analysis. Surprisingly, it was found that there was a decrease over time in the abundance of two significant families known to characterize dysbiosis, namely Enterococcaceae (phylum Firmicutes) and Enterobacteriaceae (phylum Proteobacteria). The comparison between the abundance of said families at day +3 versus day -1 in fecal samples of healthy HSP90 plasmid-treated mice, is presented in Table 3, in which “baseMean” represents the abundance of each family in the control group (day -1), and “log2(FoldChange)” or “log2FC” indicates the change between the groups (fold enhancement/reduction), represented as the base 2 logarithm thereof. For a comparison “A vs B”, a positive log2FC value indicates a higher abundance in A than in B. Accordingly, the negative log2FC values presented in Table 3 for the two comparisons, both with an adjusted p value (padj)<0.01, indicate that HSP90 plasmid suppresses the growth of dysbiosis-related bacteria. In particular, as can be seen in Table 3, a significant reduction in the abundance of Enterococcaceae and Enterobacteriaceae at day +3 compared to their abundance at day -1, was surprisingly observed.

TABLE 3

Differential abundance analysis (DESeq2) of HSP90 day +3 vs HSP90 day -1; two significant families

| Family | baseMean | log2FC | padj |
|---|---|---|---|
| Enterococcaceae | 487.96 | -8.61 | 0.00 |
| Enterobacteriaceae | 6618.71 | -5.82 | 0.00 |

### Example 2. HSP90 Plasmid Prevents TNBS-Induced Expansion of Detrimental GI Species

The microbiota composition of TNBS-administered mice further receiving the HSP90 plasmid (Group #5) was compared to that of control TNBS- administered mice receiving saline buffer (Group #3) or empty plasmid (Group #4). The bacterial taxonomy of interest in this study was as follows: Phylum > Family > Genus.

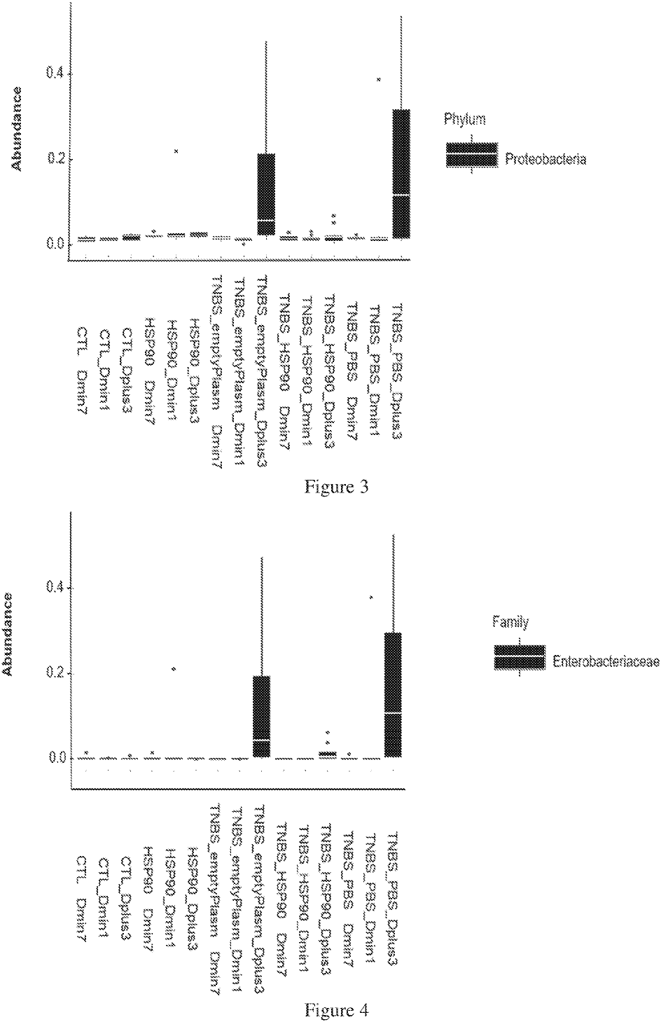

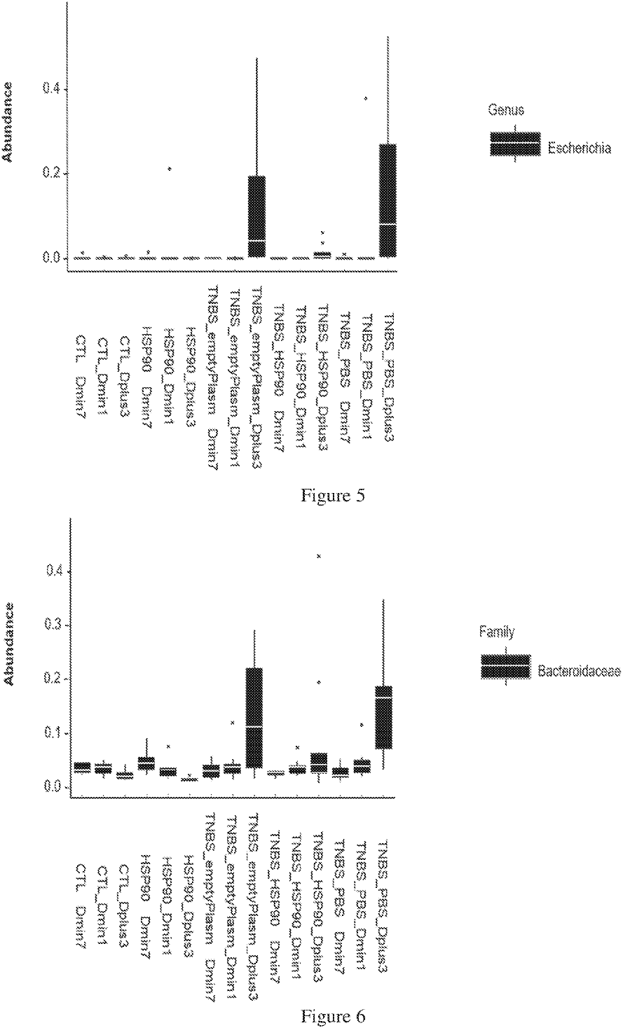

Remarkably, comparative statistical analysis of the 16S data revealed that treatment with HSP90 plasmid prevented the expansion of phylum proteobacteria (FIG. 3), particularly family Enterobacteriaceae (FIG. 4) and more particularly genus Escherichia (FIG. 5) in mice treated with TNBS at Day 3 post TNBS administration (TNBS_HSP90_Dplus3) in a selective manner, as compared to the TNBS control groups (TNBS_PBS_Dplus3 and TNBS_emptyPlasm_Dplus3) in which a significant proliferation of the respective microbiota was observed at day +3. Notably, these specific phylum, family and genus are known to expand during gut dysbiosis.

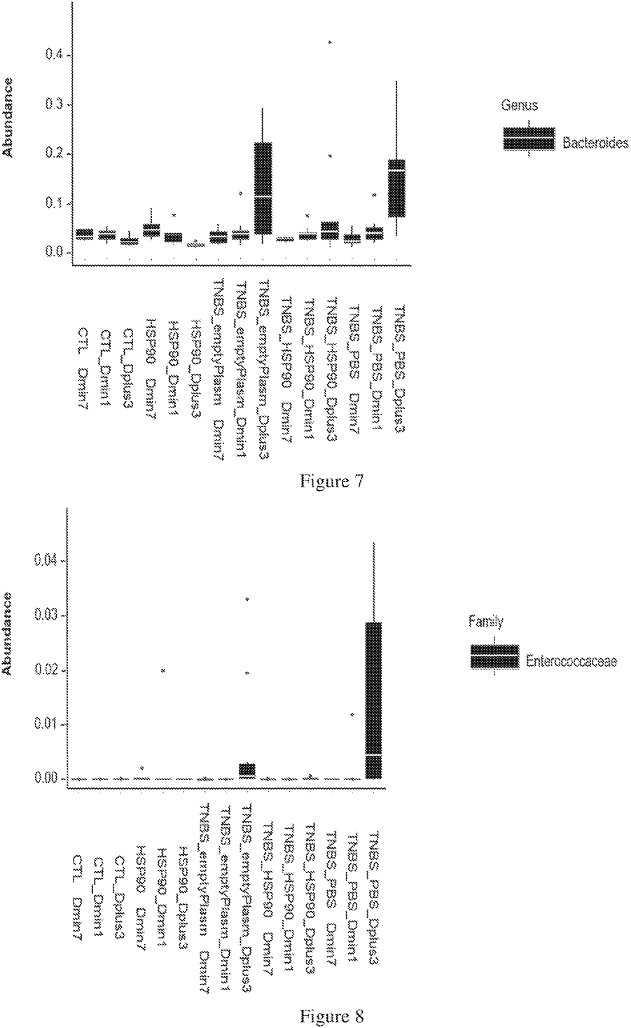

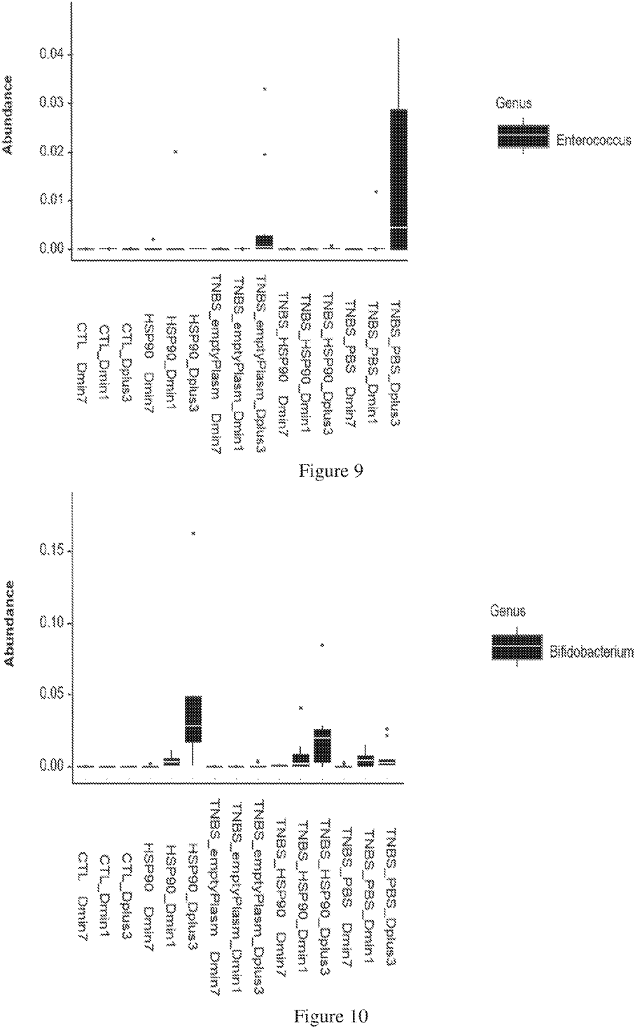

Further, HSP90 plasmid prevented the expansion of additional bacteria including those belonging to the Bacteroidaceae family (phylum Bacteroidetes), Bacteroides genus (family Bacteroidaceae), Enterococcaceae family (phylum Firmicutes), and Enterococcus genus (family Enterococcaceae), as can be seen in FIGS. 6, 7, 8 and 9, respectively.

The differences discussed hereinabove between the HSP90 plasmid-treated TNBS group and the control TNBS groups at day 3, are all characterized by adjusted p-value (padj) less than 0.05.

This can also be seen in Table 4 below, summarizing the differential abundance analysis of proteobacteria between days +3 and -1 in TNBS-treated mice. As can be seen, the TNBS-induced enhancement in Enterobacteriaceae counts (log2FC of 9.85 in the control group) was significantly reduced in the presence of the HSP90 plasmid to 6.71 log2FC, namely a reduction of 3.14 log2FC.

TABLE 4

Effect of HSP90 on TNBS-induced dysbiotic Proteobacteria expansion

| | Family | baseMean | log2FC | padj |
|---|---|---|---|---|
| HSP90 | Enterobacteriaceae | 6618.71 | -5.82 | 0.00 |
| TNBS + control plasmid | Enterobacteriaceae | 6618.71 | 9.85 | 0.00 |
| TNBS + HSP90 plasmid | Enterobacteriaceae | 6618.71 | 6.71 | 0.00 |

Overall, these results demonstrate a protective role of HSP90 plasmid against alterations of the microbiota that are a signature of dysregulated host-microbes symbiosis, supporting the use of HSP90 plasmid for GI microbiota modulation and treating or preventing dysbiosis.

### Example 3. HSP90 Plasmid Promotes the Growth of Beneficial GI Microbial Strains

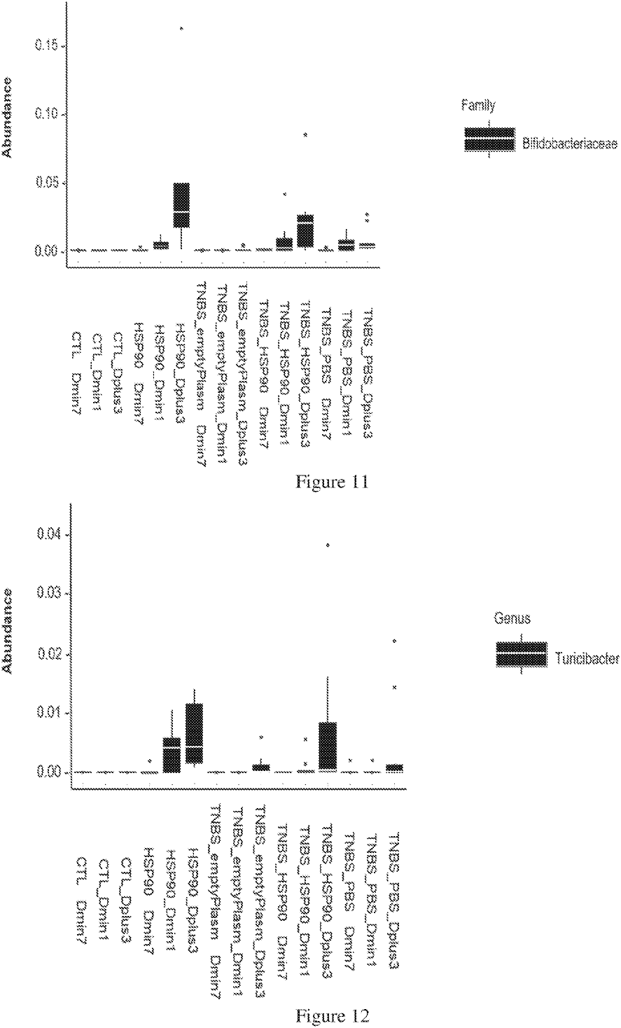

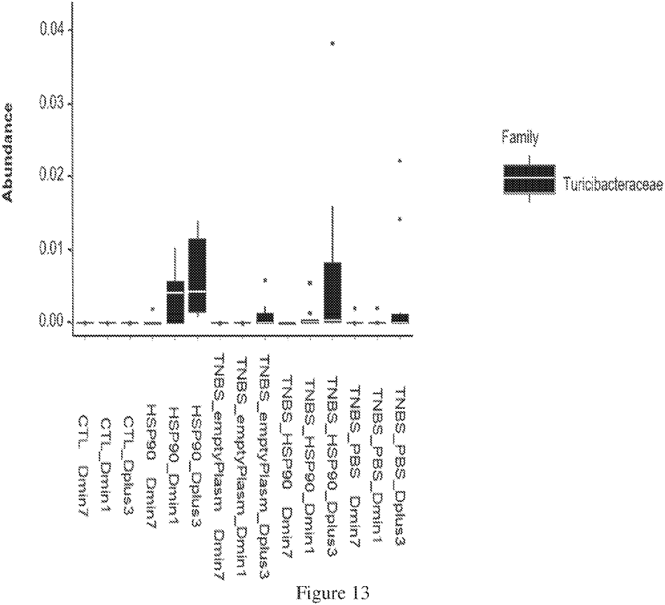

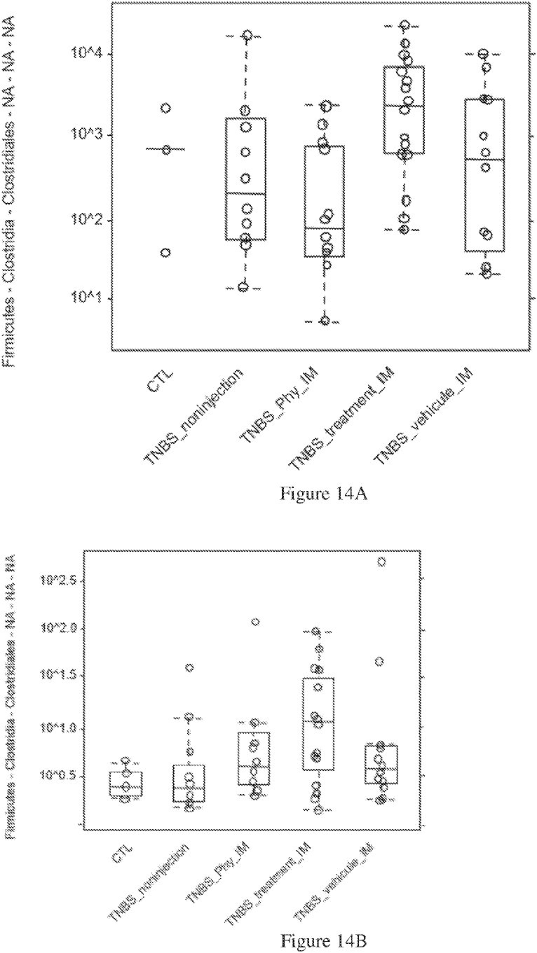

Comparative statistical analysis of the 16S data as described above further showed that the HSP90 plasmid also enhanced selectively the relative abundance of Bifidobacterium, particularly Bifidobacteriaceae, and Turicibacter, particularly Turicibacteraceae (FIGS. 10-13, respectively), both in healthy mice and TNBS-administered mice.

These findings were further confirmed in a separate experiment performed in additional groups of C57BL/6 mice (hereinafter “second experiment”), essentially as described above. The experiment included the following groups: no treatment (CTL, N=5), TNBS-treated only (TNBS-noinjection, N=12), TNBS + saline injection (TNBS-Phy/IM, N=12), TNBS + control vector (TNBS-vehicle/IM, N=12), and TNBS + HSP90 plasmid (TNBS-treatment/IM (N=16). Feces samples were collected on day +3, at sacrifice.

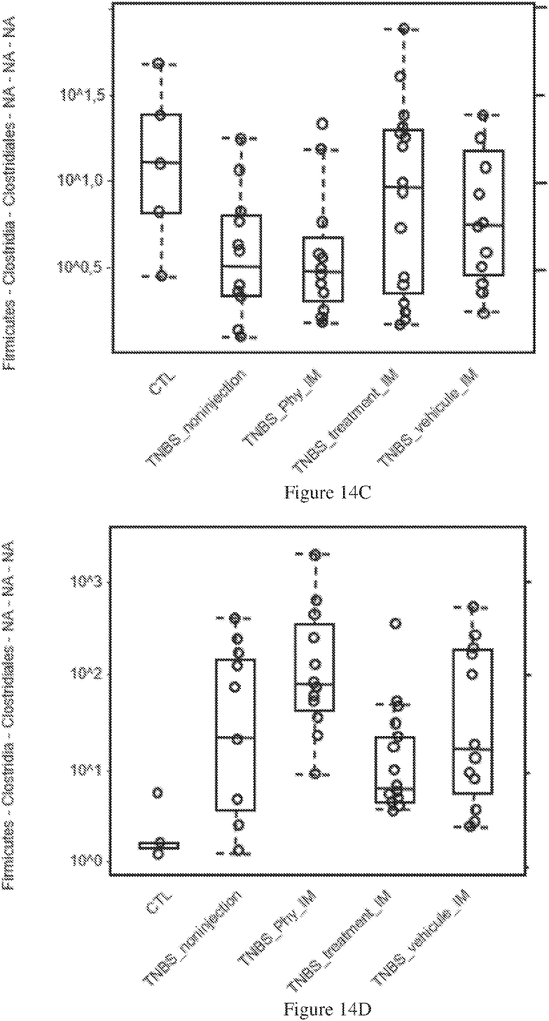

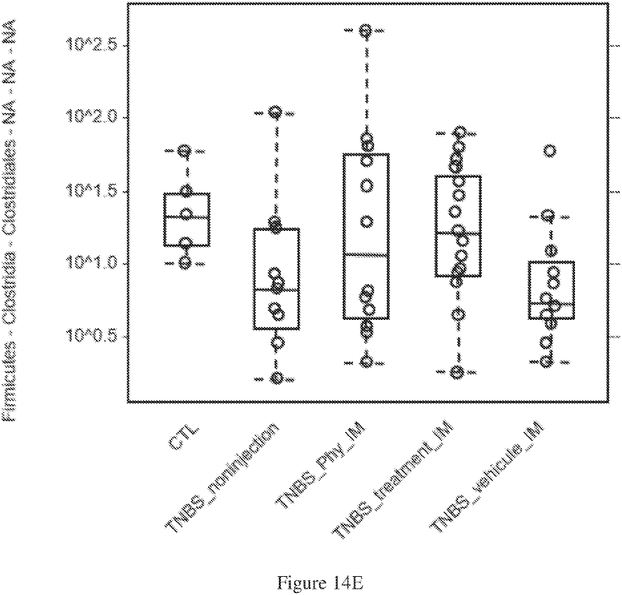

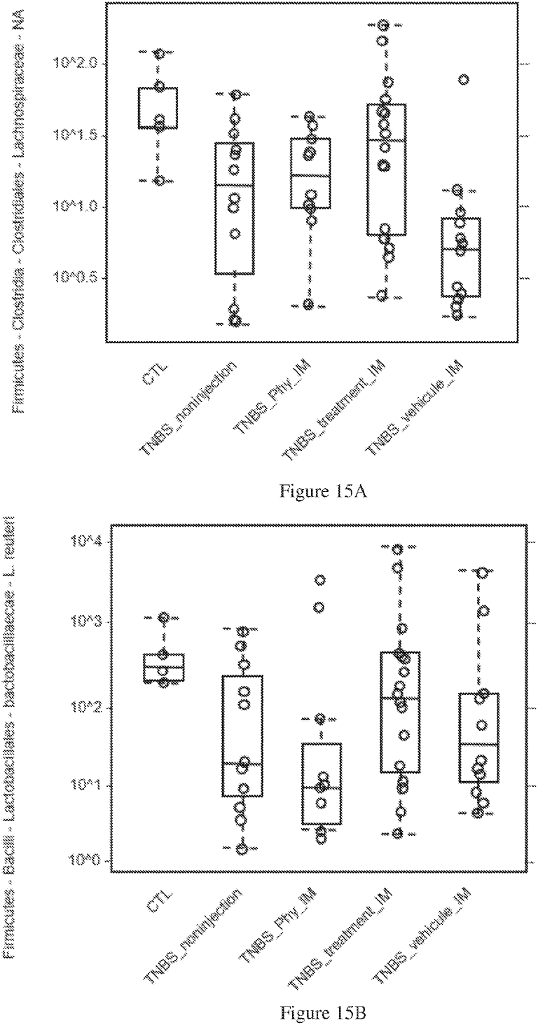

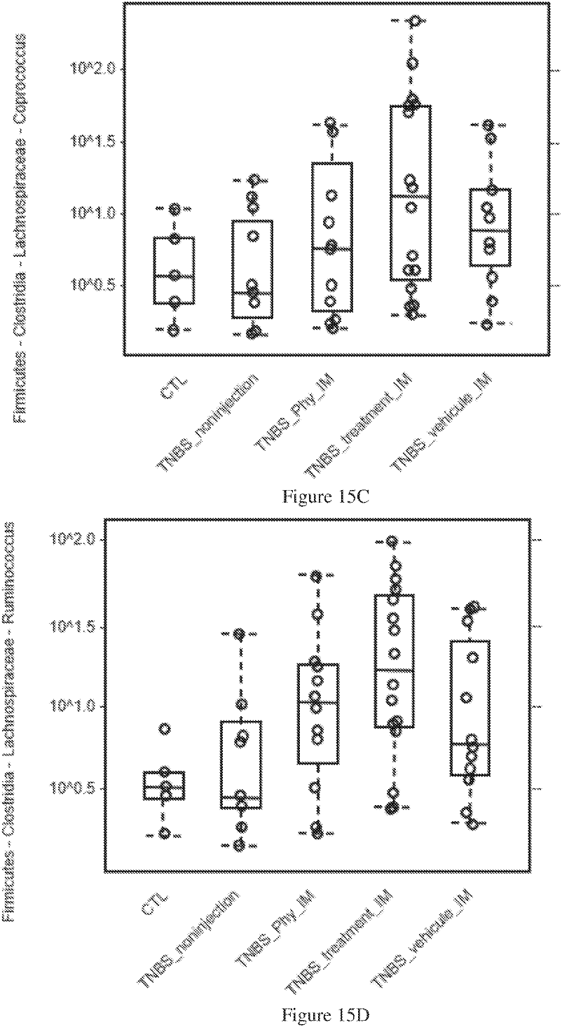

As can be seen in FIGS. 14A-14E and 15A-15D, an increase (or inhibition in TNBS-induced reduction) in bacterial strains known to exert beneficial effects in the context of gastrointestinal inflammation and/or gut barrier integrity, including Firmicutes (e.g. Lachnospiraceae spp., Lactobacillus spp., and Clostridiales spp.), Coprococcus spp., Ruminococcus spp., Bifidobacteriaceae (Bifidobacterium) spp. and Turicibacteraceae (Turicibacter) spp., was observed. Specifically, the boxplots in FIGS. 14A-14E and in FIGS. 15A-15D represent significant differences at the OTU level between the five study groups.

Thus, HSP90 treatment promoted OTUs belonging to Firmicutes and butyrate-producer bacteria, known to ameliorate mucosal inflammation and oxidative status, and reinforce the epithelial defense barrier.

### Example 4. Specific Modulation of TNF-α Secretion

A colon specimen located precisely 4 cm above the anus, was collected from mice of the different treatment groups, and used for evaluation of RNA expression levels of various cytokines. Total RNA was isolated from mouse colon tissue followed by reverse transcription for cDNA generation, according to standard procedure. The expression levels of murine cytokines were determined by measuring their cDNA levels relative to the housekeeping gene RPLP0 cDNA levels by quantitative real-time PCR using TaqMan™ assays, according to the manufacturer’s protocol.

The results indicated an unexpected specific modulation of tumor necrosis factor alpha (TNF-α) levels by the HSP90 plasmid.

Specifically, following administration of TNBS, TNF-α expression increased by 6-fold (“TNBS + saline buffer” compared to healthy “no treatment” controls). Remarkably, a 60% decrease in the relative expression level of TNF-α was observed in the “TNBS + HSP90 plasmid (TNBS-treatment/IM)” group, which significantly exceeded the reduction observed in the “TNBS + control vector (TNBS-vehicle/IM)” group. Notably, the HSP90 plasmid did not affect the levels of other cytokines, such as TGFβ.

Thus, treatment by an HSP90-encoding construct provides for a dual role in inhibiting dysbiosis and downregulating TNF-α expression. Accordingly, the treatment may be particularly useful in the treatment of patients suffering from (or at risk for developing) dysbiosis that would also benefit from TNF-α inhibition, such as subjects receiving TNF-α antagonists or inhibitors or subjects exhibiting resistance to immunomodulatory treatment (e.g. by immunosuppressants or by TNF-α antagonists or inhibitors).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1-54. (canceled)

55. A method of modifying gastrointestinal (GI) microbiota profiles in a subject in need thereof, comprising administering to the subject a nucleic acid construct comprising a nucleic acid sequence encoding a mammalian heat shock protein 90 (HSP90), or an active fragment thereof, wherein the nucleic acid sequence is operatively linked to one or more transcription control sequences, to thereby enhance the abundance of beneficial microbiota, reduce the abundance of detrimental microbiota, and/or modify microbiota biodiversity in the GI tract of said subject.

56. The method of claim 55, wherein the subject exhibits dysbiosis of the GI tract.

57. The method of claim 55, wherein the detrimental microbiota comprise at least one pathogen species belonging to the Bacteroidaceae, Enterobacteriaceae and/or Enterococcaceae family.

58. The method of claim 57, wherein said detrimental microbiota comprise at least one Enterococcus species, at least one Escherichia species and at least one Bacteroides species.

59. The method of claim 57, wherein the beneficial microbiota comprise at least one Lachnospiraceae, Lactobacillus, Clostridiales, Coprococcus, Ruminococcus and/or Turicibacter species.

60. The method of claim 55, for modifying microbiota biodiversity in the GI tract.

61. The method of claim 60, wherein the subject is afflicted with infection by drug-resistant bacteria.

62. The method of claim 61, wherein the drug-resistant bacteria are selected from the group consisting of Enterococcus, Escherichia and Bacteroides species.

63. The method of claim 55, wherein said disease or disorder is a non-autoimmune inflammatory disorder of the GI tract.

64. The method of claim 55, wherein said subject is afflicted with gut barrier dysfunction.

65. The method of claim 55, wherein said subject is afflicted with a GI disorder selected from the group consisting of: irritable bowel syndrome (IBS), celiac disease, small intestinal bacterial overgrowth (SIBO), leaky gut syndrome, and diverticular disease.

66. The method of claim 55, wherein the construct is administered in combination with at least one antibiotic, probiotic or prebiotic agent.

67. The method of claim 55, wherein said construct encodes human HSP90 alpha.

68. The method of claim 55, wherein said construct is administered in the form of a pharmaceutical composition further comprising a pharmaceutically acceptable carrier, excipient and/or diluent.

69. The method of claim 55, wherein the construct is administered in the form of a naked DNA.

70. The method of claim 55, wherein said construct is administered by intramuscular injection.

71. The method of claim 55, wherein said subject is further afflicted with a disease or condition resistant to an immunomodulatory treatment selected from immune suppressive treatment and anti-cytokine immunomodulatory treatment.

72. The method of claim 55, wherein said construct is administered in concurrent or sequential combination with a TNF-α antagonist or inhibitor.

73. The method of claim 72, wherein the TNF-α antagonist or inhibitor is selected from the group consisting of adalimumab, certolizumab, certolizumab pegol, golimumab, infliximab and etanercept.

74. A method of treating or preventing gut barrier dysfunction in a subject in need thereof, comprising administering to the subject a nucleic acid construct comprising a nucleic acid sequence encoding a mammalian heat shock protein 90 (HSP90), or an active fragment thereof, wherein the nucleic acid sequence is operatively linked to one or more transcription control sequences, to thereby decrease intestinal permeability in said subject.
